(19) United States
(12) Patent Application Publication
(10) Pub. No.: US 2004/0002121 A1
(43) Pub. Date: Jan. 1, 2004

(54) High throughput methods and devices for assaying analytes in a fluid sample

(76) Inventors: Jeffrey F. Regan, Fremont, CA (US); Randall Miller, St. Helena, CA (US); Carl Peck, Rockville, MD (US)

Correspondence Address: Peters, Verny, Jones & Schmitt, L.L.P., Suite 6, 385 Sherman Avenue, Palo Alto, CA, 94306, US

(21) Appl. No.: 10/287,765

(22) Filed: Nov. 4, 2002

Related U.S. Application Data
(60) Provisional application No. 60/337,746, filed on Nov. 6, 2001.
(60) Provisional application No. 60/396,924, filed on Jul. 17, 2002.

(51) Int. Cl. G01N 33/53; G01N 33/567; G01N 33/543

(52) U.S. Cl. 435/7.2; 436/518

(57) ABSTRACT

Methods and devices are provided herein that are useful for qualitatively detecting the presence of, and/or quantitatively determining the amount of, one or more analytes in a relatively small microvolume fluid sample, including body-fluid samples and non-body fluid samples. High throughput techniques are also provided for evaluating multiple samples.

### CROSS-REFERENCE TO RELATED APPLICATIONS

This application is related to, and claims the benefit of, U.S. Provisional Patent Application Nos. 60/337,746 filed Nov. 6, 2001, and 60/396924 filed Jul. 17, 2002, the disclosures of which are incorporated herein by reference in their entirety.

### BACKGROUND

A variety of diagnostic devices have been developed for the detection of an analyte of interest in a sample. In those devices in which sample collection and testing functions are non-linked, the transfer of collected sample to testing apparatus introduces a potential source of error and a degree of sample loss that is significant for small volume or microvolume (<200 microliters) samples. In those devices in which sample collection and testing functions are linked, the devices are dedicated in their entirety to the detection of a particular analyte, are not easily adaptable to a wide range of analytes for detection and generally require processing a single sample at a time, with no provision for high throughput analysis. Accordingly, there is a need to overcome these two limitations associated with prior art devices. The present invention satisfies this need and provides related advantages as well.

### SUMMARY OF THE INVENTION

One embodiment of the present invention is a method for detecting one or more analytes. A predetermined microvolume of a fluid sample comprising the one or more analytes is collected on a microplatform housed within a microdevice. The microplatform is dried and the presence and/or amount of one or more analytes on or from the microplatform are detected. The presence or amount thereof indicates the presence or amount of the one or more analytes.

Another embodiment of the present invention is a method for detecting one or more analytes in a fluid sample. A predetermined microvolume of the fluid sample is collected into a microdevice. At least the one or more analytes are transported within the microdevice to a microplatform housed within the device so that a predetermined volume of a fluid comprising the one or more analytes is collected at the microplatform. In this manner either the original fluid sample or a fluid sample derived from the original fluid sample is metered to the microplatform. The microplatform is dried. The presence or amount of the one or more analytes is detected either on the microplatform or off the microplatform. The presence or amount indicates the presence or amount of the one or more analytes in the fluid sample.

Another embodiment of the present invention is a method for detecting one or more analytes in a plurality of fluid samples. For each fluid sample at least one predetermined microvolume of a fluid that comprises the one or more analytes is collected at a microplatform housed in a microdevice. Each of the microplatforms is placed in a solid surface device comprising a plurality of receiving elements, each adapted to receive one of the microplatforms.

The presence and/or amount of the one or more analytes from each of the microplatforms are detected and the presence and/or amount thereof are related to the presence and/or amount of the one or more analytes in each of the fluid samples.

Another embodiment of the present invention is a method for detecting one or more analytes in a plurality of fluid samples. For each sample at least the one or more analytes are transported within the microdevice to a microplatform housed within the device so that a predetermined volume of a fluid comprising the one or more analytes is collected at the microplatform. In this manner either the original fluid sample or a fluid sample derived from the original fluid sample is metered to the microplatform. Then, each of the microplatforms is placed in a solid surface device comprising a plurality of receiving elements, each adapted to receive one of the microplatforms. The presence or amount of the one or more analytes from each of the microplatforms is detected either on or off the microplatform. The presence or amount thereof is related to the presence or amount of the one or more analytes in each of the fluid samples.

Another embodiment of the present invention is a microdevice comprising a microplatform housing, optionally a sample collection and transporting element associated with the microplatform housing and a microplatform disposed within the microplatform housing. The microplatform housing is adapted such that at least a portion thereof mates with a receiving element in a solid surface device comprising a plurality of receiving elements. Where the microdevice comprises a sample collection and transporting element, the microplatform is disposed in said microdevice for initiation of fluid communication with the transporting element.

Another embodiment of the present invention is an analytical collection and transportation system comprising a microdevice as described above and a tray comprising one or more recessed areas for housing one or more of the holding elements optionally with the microdevice releasably attached thereto. The analytical collection and transportation system may further comprise a solid surface device comprising a plurality of receiving elements for receiving the microplatform housing.

### BRIEF DESCRIPTION OF THE DRAWINGS

FIG. 1A is perspective view of a patient's arm with a drawn blood drop.

FIG. 1B is a perspective view of a device in accordance with the present invention.

FIG. 1C is an expanded view of a portion of the device of FIG. 1B.

FIG. 1D is perspective view the device of FIG. 1B with a cap being attached at an end thereof.

FIG. 1E is perspective view of a tray with a plurality of recesses for receiving devices of FIG. 1B.

FIG. 1F is perspective view of a solid surface device comprising a plurality of receiving elements designed to receive a microdevice portion of a device of FIG. 1B.

FIG. 1G is an expanded view of a portion of the device of FIG. 1F having a microdevice portion of a device of FIG. 1B seated therein.

FIG. 2A is a perspective view of a microdevice in accordance with the present invention.

FIG. 2B is another embodiment of a microdevice in accordance with the present invention in a cross-sectional view including within it the microdevice of FIG. 2A.

FIG. 2C is a cross-sectional view of the microdevice of FIG. 2A seated in a receiving element of a solid surface device.

FIG. 3A is a perspective view of another embodiment of a device in accordance with the present invention.

FIG. 3B is a perspective view of the device of FIG. 3A above a tray comprising a plurality of recesses for receiving the device of FIG. 3A.

FIG. 3C is perspective view of a solid surface device comprising a plurality of receiving elements designed to receive a microdevice portion of a device of FIG. 3A.

FIG. 3D is perspective view of a solid surface device comprising a plurality of receiving elements wherein one of said receiving elements has a microdevice portion of the device of FIG. 3A seated therein.

FIG. 4A is a perspective view of another embodiment of a device in accordance with the present invention together with a tray for housing the device wherein the tray has a plurality of elements for storing unused microdevice portions of the device and a plurality of elements for storing used microdevice portions of the device.

FIG. 4B is a perspective view of the device of FIG. 4A with a microdevice attached to an end thereof together with the tray of FIG. 4A.

FIG. 4C is perspective view of the device of FIG. 4A that has deposited a used microdevice into an element of the tray of FIG. 4A.

FIG. 4D is perspective view of a solid surface device comprising a plurality of receiving elements for receiving a microdevice portion of the device of FIG. 4A.

FIG. 5 is a graph depicting the correlation between assay results from the method of the invention compared to assay results obtained without collection on the device of the invention as in Example 1.

FIG. 6 is a graph depicting a membrane based standard curve from density analysis of photograph of microplatforms of the present invention that were used in Example 2, Part A, for the detection of LH.

FIG. 7 is a graph depicting the results of an assay of LH serum samples conducted in accordance with the present invention and the results of a known assay (Abbott AxSym®, Abbott Laboratories, Abbott Park Ill.) performed on the same LH serum samples (Example 2, Part B).

FIG. 8 is a graph depicting the results of an assay for glucose (Example 3) where glucose samples were collected on a membrane and calorimetric detection was carried out by post-collection enzyme addition.

FIG. 9 is a graph depicting a membrane based standard curve from density analysis of photograph of microplatforms of the present invention that were used in Example 4 for the detection of digoxin.

FIG. 10 is a graph depicting the correlation between serum cortisol assay results from the method of the invention compared to assay results obtained without collection on the device of the invention as in Example 1.

FIG. 11 is a graph depicting the correlation between serum cortisol assay results derived from whole blood samples utilizing the method of the invention compared to assay results obtained without collection on the device of the invention as in Example 1.

FIG. 12 is a graph depicting the correlation between serum cortisol assay results derived from whole blood samples utilizing the method of the invention compared to assay results obtained without collection on the device of the invention as in Example 1.

### DETAILED DESCRIPTION OF THE INVENTION

As used herein, the phrase “fluid-sample” refers to either a “body-fluid sample” or a “non-body-fluid sample.” The phrase “body-fluid sample” refers to any fluid obtained from the body of a mammal (e.g., human, monkey, mouse, rat, rabbit, dog, cat, sheep, cow, pig, and the like), bird, reptile, amphibian or fish that is suspected of containing a particular target analyte or analytes to be detected. Exemplary body-fluid samples for detection herein can be selected from one or more of whole-blood, plasma, serum, interstitial fluid, sweat, saliva, urine, semen, blister fluid, inflammatory exudates, and the like. Also explicitly contemplated herein as a “body-fluid sample” are body-gas and body-vapor. The phrase “non-body-fluid sample” refers to any fluid not obtained from the body of a mammal, bird, reptile, amphibian or fish, which is suspected of containing a particular target analyte or analytes to be detected. Exemplary non-body-fluid samples include cell-culture media, artificial-collection-fluid, dialysate (see, e.g., Forest, et al., 1997, The Drug Monit, 19(1):74-78), and the like. An artificial-collection-fluid (or extraction fluid) can be prepared by bathing a particular surface area of an animal or an inanimate object with a fluid to collect into the fluid an endogenous or exogenous analyte for detection.

As used herein, the term “analytes” or grammatical variations thereof, refers to any substance being measured in the methods provided herein. Exemplary substances for detection and/or measurement herein include, but by no means is limited to, peptides, proteins (including enzymes, tumor markers, antibodies, antigenic proteins, glycoproteins, lipoproteins and avidin), hormones (such as thyroxine, triiodothyronine, human chorionic gonadotropin, estrogen, adrenocorticotrophic hormone “ACTH” and substance P), vitamins, human immune system modulators (such as interleukin-6), steroids, carbohydrates (such as polysaccharides), glycolipids, drugs (such as digoxin, phenytoin, phenobarbital, morphine, carbamazepine and theophylline), antibiotics (such as gentamycin), components of cells and infectious agents (such as Streptococcal species, herpes viruses, Gonococcal species, Chlamydial species, retroviruses, influenza viruses, Prevotella species, Porphyromonas species, Actinobacillus species and Mycobacterium species), nucleic acids (including single- and double-stranded oligonucleotides), pharmaceuticals, haptens, lectins, biotin, a thyronine derivative (such as thyroxine and triiodothyronine), morphine, theophylline, vancomicin, tobramicin, TSH, human chorionic gonadotrophin hormone (“hCG”), immunoglobulin M (“IgM”), immunoglobulin G (“IgG”), immunoglobulin E (“IgE”) and immunoglobulin A (“IgA”), creatine kinase-MB, troponins T and I, and apoproteins A, Al and B, and the like.

The analytes include exogenous or environmental toxins such as pesticides, mercury, chemical warfare agents, bioterrorism agents, and so forth. Among pesticides of interest are polyhalogenated biphenyls, phosphate esters, thiophosphates, carbamates, polyhalogenated sulfenamides, dioxins, organophosphate insecticides, their metabolites and derivatives.

The microdevice of the invention is generally a structure that functions to collect or take-up a body-fluid sample from a subject or a non-body-fluid sample from its location, and migrate the fluid sample within the device to a particular location, without any direct human manipulation of the sample (e.g., aliquoting, and the like), and optionally meter a precise amount of fluid, such that a detection reagent can be used to determine the presence and/or amount of a particular analyte or analytes in the fluid sample either at the particular location or on an extract from the particular location. In certain embodiments, the microdevice comprises a single mechanism for both collecting and transporting a fluid sample within the microdevice; a microplatform; optionally a capture-moiety attached to the microplatform; and a housing in which each of the components resides.

“Optional” or “optionally” means that the subsequently described event or circumstance or element may or may not occur or be present, and that the description includes instances where said event or circumstance or element occurs or is present and instances in which it does not. For example, “optionally a capture-moiety attached to the microplatform” means that the capture-moiety may or may not be present on the microplatform.

In another embodiment, the microdevice comprises a mechanism for collecting a fluid sample into the microdevice, a separate mechanism for transporting a fluid sample within the microdevice to a particular locus; a microplatform; optionally a capture-moiety attached to the microplatform; and a housing in which each of the components resides. In one embodiment, when the body-fluid sample is whole-blood, the microdevice optionally further includes a cell separating mechanism so that blood serum or plasma is delivered to a particular locus on the microplatform housed within the device. In yet another embodiment, a harvesting mechanism, such as a lancet or needle including a microneedle, used for making the body-fluid-sample available for collection, is integrated with the microdevice such that the harvesting and subsequent collection of the body-fluid sample into the microdevice are completed using a single structure instead of two separate structures. A microneedle is a needle that has a capacity to take up a microvolume of fluid. The harvesting mechanism may be integrated with the microdevice permanently or non-permanently, i.e., fixedly or detachably.

The size of the microdevice will vary from micro-scale up to macro-scale depending on the size of the microplatform utilized within. Typically, the size will range anywhere from about 1 micron×1 micron×1 micron up to 5 cm×5 cm×15 cm. For example, in a particular macro-scale embodiment contemplated herein, the size of the microdevice is 1×1×7 cm.

In one embodiment, the microdevice further contains a region thereon that permits the encoding of patient and sampling information on each device. Such encoding can be accomplished using handwriting; a barcode or similar optical method; radiofrequency; electronic storage for IR or electronic downloading; and/or can be auto time stamped to indicate the precise time of the blood draw.

The microplatform of the present devices is a material housed within the microdevice that functions to capture one or more analytes in a sample. The capture may be by specific binding means such as by the use of a capture moiety that is a member of a specific binding pair such as, e.g., a specific binding partner for the analyte, or by non-specific binding means such as adsorption or absorption, and the like. When a capture moiety is employed, it is generally fixed at a particular locus for subsequent capture of a target analyte. Specific binding involves the specific recognition of one of two different molecules for the other compared to substantially less recognition of other molecules. The two different molecules are termed “members of a specific binding pair.” On the other hand, non-specific binding involves non-covalent binding between molecules that is relatively independent of specific surface structures. Non-specific binding may result from several factors including hydrophobic interactions between molecules.

Members of the specific binding pair may be members of an immunological pair such as antigen-antibody, other specific binding pairs such as biotin-avidin, hormone-hormone receptors, enzyme-substrate, nucleic acid duplexes, IgG-protein A, polynucleotide pairs such as DNA-DNA, DNA-RNA, and the like.

The microplatform may be, for example, a membrane, a filter, a plastic support, a silicon support, a glass support, and the like. The shape of the support material is not critical. It can, for example, be a flat or planar surface such as a square, rectangle, oval or circle; a curved surface; or a three-dimensional surface such as a particle including bead, strand, precipitate, tube, sphere, torus, cube, and the like. Any compatible support can be used as a microplatform in conjunction with the methods described herein. Exemplary support materials for microplatforms for use herein can be selected from one or more of paper, such as filter paper; diazotized cellulose; filters, such as nitrocellulose filters; membranes, such as nylon membranes; organic or inorganic materials or combinations thereof, including plastics, such as polypropylene, PVC, or polystyrene; ceramic; silicon; (fused) silica; silicon-derivatives (e.g., PEO-modified silicon; Sofia et al., Marcomolecules, 31:(15)5059-5070, (1998)), and the like; quartz; glass, which can have the thickness of, for example, a glass microscope slide or a glass cover slip; gold (see, e.g., gold cantilever described in Wu et al., 2001, Nature Biotech., 19:856-860); polyacrylamide or other type of gel pad, e.g., an aeropad or aerobead, made of an aerogel (a highly porous solid), including a film prepared by drying of a wet gel, and the like. An exemplary glass is, e.g., agarose coated glass described in Afanassiev et al., 2000, NAR, 28(12):E66; covalently modified glass, such as siliconized-glass described in Eckerskorn et al., 1988, Eur. J. Biochem, 176(3):509-519; polyelectrolyte-treated glass described in Wang et al., 1998, J. Chromatorgraphy A., 808(1-2):61-70. Another exemplary silicon is untreated silicon described in Coen et al., 2001, J. Colloid, Interface Sci., 15;233(2):180-189.

The dimensions of a substantially circular or oval microplatform may be about 1 to about 10 mm, about 2 to about 8 mm, about 3 to about 7 mm, about 4 to about 6 mm and so forth in diameter. For a substantially rectangular or square microplatform, the dimensions are about 1 mm to about 10 mm in length by about 1 mm to about 1 mm in width, about 2 mm to about 8 mm by about 2 mm to about 8 mm, about 3 mm to about 7 mm by about 3 mm to about 7 mm, about 4 mm to about 6 mm by about 4 mm to about 6 mm and so forth. The microplatform has a thickness of about 1 to about 10,000 microns, about 10 to about 1,000 microns, about 50 to about 500 microns, about 75 to about 300 microns, about 100 to about 200 microns, and so forth.

The term “locus” or “loci,” or grammatical variations thereof, refers to the one or more location(s) on the microplatform where a target analyte may become specifically or non-specifically bound. In the case of specific capture there may be one or more members of specific binding pairs as capture-moieties attached thereto at which the target analyte is captured for detection. The size, shape and physical spacing of the various loci within a microplatform can be readily adapted depending on the assay format desired. Typically, the one or more loci can be of an area of about 1 micron² up to about 700 mm² When more than one locus in contained on the microplatform the loci can be spaced about 50 microns up to about 5 mm apart (center-to-center). For example, in certain embodiments the physical dimension of a particular locus or loci is planar and is about 0.001 mm² to about 3 cm². In specific embodiments a planar locus is no greater than 3000, 2500, 2000, 1500, 1000, 500, 400, 300, 200, 100, 75, 50, 40, 30, 25, 20, 15, 10, 5, 4, 3, 2, 1, 0.5, 0.1, 0.01, 0.001 mm². In other embodiments, the physical dimensions of a particular locus or loci are volumetric ranging in volume from about 1 nanoliter to about 250 microliters, or about 1 nanoliter to about 100 microliters.

In one embodiment, there is a single locus on a flat microplatform within the microdevice containing the capture-moiety. For example, in this embodiment the physical dimension of a particular locus or loci is about 0.001 mm² to about 3 cm². In specific embodiments the locus is no greater than 3000, 2500, 2000, 1500, 1000, 500, 400, 300, 200, 100, 75, 50, 40, 30, 25, 20, 15, 10, 5, 4, 3, 2, 1, 0.5, 0.1, 0.01, 0.001 mm². Although the surface area of the locus or loci can be smaller than the area of the microplatform, also contemplated herein is an embodiment where the surface area of the locus coincides with the entire surface of the microplatform, such that the surface area sizes of the locus and the microplatform are the same.

In another embodiment, multiple loci are uniformly spaced apart, such as, e.g., approximately 5 mm apart. For example, a particular microplatform within a microdevice could comprise a rectangular grid, with, for example, a 2×2 or 3×3 grid, of roughly circular spots (loci) of capture-moieties, which are, e.g., about 1 to about 5 mm² in area (e.g., about 1.5 mm in diameter) and about 300 micrometers apart. In yet another embodiment, a particular microplatform within a microdevice comprises multiple loci arranged in a 33×33 matrix within a 1 cm×1 cm section of microplatform. In one embodiment, each locus within the matrix is individually exposed to fluid-sample over time, which allows the collection of up to 1089 distinct samples over a given time period of hours, days or weeks. In another embodiment, each locus within the matrix contains a unique capture-moiety directed to a unique analyte, which allows the simultaneous analysis of up to 1089 analytes from a single fluid-sample. As set forth herein, larger and smaller loci areas and spacings are also contemplated.

Various loci within or on a microplatform can be defined by modification of the surface itself. For example, a plastic surface can comprise portions made of modified or derivatized plastic, which can serve, e.g., as sites for the addition of specific types of capture-moieties. For example, PEG can be attached to a polystyrene surface and then derivatized with carboxyl or amino groups, double bonds, aldehydes, and the like, for the attachment of various capture-moieties. Alternatively, a plastic surface can comprise molded structures such as protrusions or bumps, which can serve as platforms for the addition of capture-moieties. In another embodiment, various loci can be located within gel pads, e.g., polyacrylamide gel pads or aeropads, which are arrayed in a desired pattern on a surface such as, e.g., glass, or are sandwiched between two surfaces, such as, e.g., glass and a quartz plate. Linkers and capture-moieties can be immobilized on the surface of such pads, or can be imbedded within them. A variety of other arrangements of gel pads on surfaces will be evident to one of skill in the art, and can be produced by routine, conventional methods. The relative orientation of the various loci within the microplatform can take any of a variety of forms including, but not limited to, parallel or perpendicular arrays within a square or rectangular or other surface, radially extending arrays within a circular or other surface, or linear arrays, and the like.

As used herein, the phrase “capture-moiety” refers to any agent, protein, molecule or compound that functions to bind either specifically or non-specifically (e.g., covalently, ionically, immunologically, electrostatically, and the like) to a particular target analyte. In one embodiment, the capture-moiety is selected such that it is specific for a particular target-analyte to be detected. When more than one analyte is detected using the methods provided herein, an equivalent number of capture-moieties is needed. Exemplary capture-moieties contemplated for use herein can be selected from one or more of an anti-analyte-specific antibody, an anti-analyte-specific aptamer (see, e.g., Jayasena et al., 1999, Clin. Chem. 45(9):1628-1650), an anti-analyte-specific reactive-enzyme, an antigen for a serum antibody-target analyte, a receptor for a specific target-analyte, and the like. See also, for example the affinity molecules described in U.S. Pat. No. 5,856,092, the relevant disclosure of which is incorporated herein by reference.

The present invention has application to the detection of blood glucose. When methods and devices provided herein require organizing the microdevices onto a solid-surface for high throughput detection, the microplatform may or may not comprise a blood glucose capture-moieties. A blood glucose capture-moiety is any agent, molecule or compound that functions to bind (e.g., covalently, ionically, immunologically, electrostatically, and the like) to blood glucose. When the methods and devices provided herein are not used for high throughput detection, the microplatform does not comprise a blood glucose capture moiety.

Capture-moieties are immobilized to a microplatform using methods well-known in the art. Exemplary immobilization methods of the capture-moiety onto the solid-surface include drop-wise application, spraying, dip coating, wicking, ink-jet application, and the like. In certain embodiments prior to immobilization of the capture-moiety, the microplatform surface is prepared using, for example, stabilizers (e.g., nonspecific proteins), flow enhancers (e.g., PEG, glycerol, and the like), gel forming units (e.g., carboxymethyl cellulose), and the like. Stabilizers are helpful for drying of samples on the microplatform such as a paper microplatform and the like. Stabilizer include proteins, sugars, commercial stabilizers such as Stabilcoat® (see examples), and so forth.

As used herein the term “collecting” in the context of “collecting one or more microvolume samples of fluid from a subject directly into one or more microdevices” refers to the process of taking-up or drawing-up (i) the body-fluid sample from the subject either directly from the subject or indirectly such as sample from a sample collection device that is independent of the microdevice of the invention or (ii) the non-body-fluid sample from its location directly into the microdevice in such a way that no human manipulation of the sample is utilized. For example, the body-fluid sample can be taken up directly from any skin-surface of a particular subject using a variety of well-known methodologies, including but not limited to, capillary action (chemically aided wicking) using capillary tubes/channels or absorbent paper, applied pressure (e.g., vacuum or pump, such as a micropump or a negative pressure pump), and the like.

In certain embodiments, when the body-fluid sample is blood, the blood is made available for collection into the microdevice using any one of the well-known methods and/or devices for generating blood from any skin surface of a subject. For example, numerous methods for generating blood from a subject using a sample generating means such as, for example, a piercing device or some other type of device capable of forming an unobstructed opening in the skin of the subject are well-known in the art an include a lancet (e.g., microlancets), a microneedle (see, e.g., U.S. Pat. No. 5,928,207), and skin ablation (see, e.g., U.S. Pat. No. 6,206,841 B1). Piercing devices suitable for use in the methods and devices provided herein include, but are not limited to, mechanical lancing assemblies. The sample generating means may be separate from the present microdevice or part of the present microdevice.

In a particular embodiment, the body-fluid sample is generated for collection into the microdevice by a lancet (including microlancets). Mechanical lancing assemblies are well-known in the art. These assemblies include standard steel lancets, serrated devices, and multiple tip devices. The lancets can be made from metal or plastic. Multiple tip devices provide redundancy, which can reduce the number of failures and increase the volume of blood extracted. Exemplary lancing assemblies suitable for use in the methods and devices provided herein, include those described in U.S. Pat. Nos. Re. 32,922, 4,203,446, 4,990,154, and 5,487,748, all of which are incorporated herein by reference. In certain embodiments, if a vacuum is employed, the lancing assembly should be designed so that a vacuum can be formed and drawn through the assembly. The lancing assembly can be designed to allow automatic cocking and automatic triggering of the lancet.

Typically, the lancing assembly comprises at least one lancet. Standard lancets can be used in the lancing assembly of this invention. In certain embodiments, narrow gauge (28 to 30 gauge) lancets are employed. In other embodiments, the depth of penetration of the lancet into the skin of the subject typically ranges from about 0.4 to about 2.5 mm, more preferably from about 0.4 to about 1.6 mm. Accordingly in some embodiments, the length of the lancet or lancets can range from about 1 mm to about 5 mm. The lancet of the lancing assembly can be cocked manually or automatically, e.g., by means of a vacuum-actuated piston or diaphragm. Likewise, the lancet of the lancing assembly can be triggered manually or automatically, e.g., by means of a vacuum-actuated piston or diaphragm. In particular embodiments, the lancets employed herein are 510 k approved.

Exemplary methods of ablating skin (e.g., at the finger, forearm, and the like) to form an unobstructed opening in the skin are well-known in the art and include the use of a: laser, sonication, heat, abrasions, adhesive removal of stratum corneum, hydration using fluid jets, physical piercing, and the like. Exemplary lasers suitable for forming an unobstructed opening in the skin to draw blood are well known in the art. See for example, U.S. Pat. Nos. 4,775,361, 5,165,418, 5,374,556, International Publication Number WO 94/09713, and Lane et al. (1984) IBM Research Report—“Ultraviolet-Laser Ablation of Skin,” and the like, all of which are incorporated herein by reference. Fluid jets suitable for forming an unobstructed opening in the skin employ a high pressure jet of fluid, preferably a saline solution, to penetrate the skin.

In addition, the methods of harvesting the sample of blood from the opening in the skin can be carried out using a combination of harvesting enhancing elements. Harvesting enhancing elements suitable for use in the methods provided herein include, but are not limited to, vacuum, skin stretching elements, and heating elements. When these elements are used in combination with a sample generating means, the volume of blood extracted is greatly increased, particularly when a vacuum is applied in combination with skin stretching. In this combination, the vacuum not only causes the blood to be rapidly removed from the unobstructed opening by suction, it also causes a portion of the skin in the vicinity of the opening to be stretched. Stretching of the skin can be effected by other means, such as mechanical means or adhesives. Mechanical means include devices for pinching or pulling the skin; adhesives bring about stretching of the skin by means of pulling. Like a vacuum, a heating element operates more effectively in combination with other techniques, e.g., stretching of the skin. Also contemplated is the incorporation of a lancet in the device and drawing the sample into the device in the same step as lancing. For example, the skin is lanced and a bead of blood is allowed to form on the skin. The present device is then touched to the skin at the point of the bead of blood. In another exemplary approach a lance is replaced with a hollow needle and the device is pushed into skin and blood is drawn through the needle directly into the present device.

As used herein, the phrase “microvolume samples” refers to the quantity of fluid-sample collected into the microdevice for analysis. Exemplary microvolume samples contemplated for use in the methods provided herein range from less than about 1 nanoliter to about 250 microliters or more, or from about 1 nanoliter to about 100 microliters or more. In certain embodiments, the microvolume can be an amount selected from less than: 250 microliters, 200 microliters, 150 microliters, 100 microliters, 50 microliters; 40 microliters; 30 microliters; 20 microliters; 10 microliters; 5 microliters; 1 microliter; 900 nl; 800 nl; 700 nl; 600 nl; 500 nl; 400 nl; 300 nl; 200 nl; 100 nl; 75 nl; 50 nl; 40 nl; 30 nl; 20 nl; 15 nl; 10 nl; 5 nl or 1 nl within the scope of the present invention that the microvolume may be less than 1 nanoliter and greater than 250 microliters depending on the nature of the assay, microdevice, and so forth.

As used herein, the phrase “transporting each fluid sample within the microdevice” refers to the migration of the body fluid or non-body-fluid sample from the exterior region of the microdevice that initially comes in contact with the fluid sample through the microdevice such that a fluid comprising at least the one or more analytes reaches a desired locus or loci within or on the microplatform of the microdevice. Other components of the sample may also be transported with the one or more analytes. In one example, the fluid sample can be transported within the microdevice using a variety of well-known methodologies, including but not limited to, capillary action (also referred to herein as chemically aided wicking) using capillary tubes or channels, or absorbent paper (see, e.g., U.S. Pat. No. 6,206,841 B1), hydrostatic pressure, applied pressure (e.g., vacuum or pump, such as a nano-fabricated pump), heat, electricity, and the like. Regardless of the manner in which the blood sample is collected, the sample can be analyzed immediately or at a time later than the time of collection or at a geographic location remote from the location of collection or both, as described herein.

In a particular embodiment, the fluid sample is transported or migrated within the microdevice by capillary or chemically aided wicking action. As used herein, the phrases “capillary action” or “chemically aided wicking action” refers to the movement of a fluid within the spaces of a porous material due the forces of adhesion, cohesion, and/or surface tension. Capillary or wicking action can be further characterized as either: (a) the flow of fluid along a material wherein the nature of the material itself is hydrophilic, such as, for example, cellulose or a glass capillary tube; (b) the flow of fluid along a material wherein at least one chemical substance is applied to the surface of the material, such as, for example, nylon coated with surfactant; and (c) the flow of fluid along a material that has been rendered hydrophilic by means of a chemical or physical process, such as, for example, treatment of polyester by means of corona discharge treatment, plasma treatment, flame treatment, or the like.

In certain embodiments, the blood-transporting material is preferably made from polymeric material, cellulosic material, natural fibrous material, or an equivalent material. Exemplary polymeric materials suitable for the blood-transporting material include, but are not limited to, polymers comprising amide monomeric units, e.g., nylon, ester monomeric units, alkylene monomeric units, e.g., polypropylene, polyethylene, cellulosic monomeric units, and combinations thereof. In other embodiments, the blood-transporting material can be a mesh. One type of mesh can be constructed of finely woven strands of polymeric material; however, any woven or non-woven material may be used, provided that the blood-transporting material transports the blood to the desired locus before the blood evaporates or clots.

In certain embodiments, a fine mesh suitable for the methods and devices provided herein has a percent open area of from about 40 to about 45%, a mesh count of from about 95 to about 115 fibers per cm, a fiber diameter of from about 20 to about 40 μm, and a thickness of from about 40 to about 60 μm. A particular fine mesh for use herein is NY64 HC mesh, available from Sefar (formerly ZBF), CH-8803, Ruschlikon, Switzerland. Another type of mesh contemplated for use herein is a coarse mesh that has a percent open area of from about 50 to about 55%, a mesh count of from about 45 to about 55 fibers per cm, a fiber diameter of from about 55 to about 65 μm, and a thickness of from about 100 to about 1000 μm. A particular coarse mesh is NY151 HC mesh, available from Sefar (formerly ZBF), CH-8803, Ruschlikon, Switzerland. Additional mesh characteristics are further exemplified in U.S. Pat. No. 5,628,890, the relevant disclosure of which is incorporated herein by reference.

The purpose of the at least one chemical substance applied to the surface of the material of the blood-transporting material, as set forth above, is to promote the flow of fluid along the surface of the material. Chemical substances suitable for this purpose include the well-known surfactants. Surfactants reduce the surface tension of the surface upon which they are coated and allow the coated surface to attract rather than repel fluids. A commercially available surfactant suitable for use in the methods and devices provided herein is a fluorochemical surfactant having the trade designation “FC 170C FLUORAD”, available from Minnesota Mining and Manufacturing Company, St. Paul, Minn. This particular surfactant is a solution of a fluoroaliphatic oxyethylene adduct, lower polyethylene glycols, 1,4-dioxane, and water. In certain embodiments, approximately 1 to 10 μg surfactant per mg of blood-transporting material is employed. The particular surfactant loading varies depending upon the nature of the material used to transport the blood (the blood-transporting material) and the particular surfactant used. The appropriate amount can readily be determined empirically by observing flow of sample along the blood-transporting material with different levels of surfactant loading. In other embodiments, the surfactant may not be necessary if the mesh is made of hydrophilic material.

The blood-transporting material is capable of allowing a sufficient amount of blood to uniformly flow through it at a rate such that a sufficient amount of blood, e.g., from about 1 nl to about 100 μl, reaches the locus having the capture-moiety therein for the desired reading of the analyte level.

In some embodiments the dimensions of a substantially circular or oval blood collection and transporting member may be about 1 to about 10 mm, about 2 to about 8 mm, about 3 to about 7 mm, about 4 to about 6 mm and so forth in diameter. For a substantially rectangular or square blood collection and transporting member, the dimensions are about 1 mm to about 10 mm in length by about 1 mm to about 1 mm in width, about 2 mm to about 8 mm by about 2 mm to about 8 mm, about 3 mm to about 7 mm by about 3 mm to about 7 mm, about 4 mm to about 6 mm by about 4 mm to about 6 mm and so forth. The blood collection and transporting member usually has a thickness of about 1 to about 10,000 microns, about 10 to about 1,000 microns, about 100 to about 500 microns, about 200 to about 300 microns, and so forth.

In one embodiment, when the body-fluid sample is whole blood, a predetermined microvolume of whole blood is taken into the microdevice by one the aforementioned methods. The whole-blood sample is transported through a cell separating mechanism so that blood serum is delivered to the microplatform either at a particular locus or on or within the entire microplatform or a portion thereof. The microplatform collects a predetermined microvolume of the blood serum, which comprises one or more analytes to be determined. As used herein, the phrase “cell separating mechanism” refers to any device or means that functions to separate cells from serum or plasma in whole blood. Exemplary means are well-known in the art and include capillary devices (e.g., glass tubes, membranes, absorbent paper), membranes, centrifugation, and the like. Exemplary cell separating membranes include: Primecare Blood Separation membrane codes S/G, C/Q, C/S, and X (commercially available from Spectral Diagnostics, Whitestone, Va.); CytoSepMedia Grades 1660, 1662, 1663; Hemasep L medium (commercially available from Pall Corp., Port Washington, N.Y.); Accusep (commercially available from Schleicher & Schuell, Keene, N.H.); and the like. For the separation of plasma, an anti-coagulant may also be used with the blood-transporting material.

In accordance with the methods provided herein, the analytes are typically detected by adding a detection-reagent (also referred to herein as a detection-substrate) to the microplatform having the capture moiety:target analyte complex therein or to an extract from the microplatform. In one approach the detection-reagent can be applied such that it does not move laterally through the microplatform. The detection-reagent can also be added to the microplatform at a different geographic location than the location of sample collection. Exemplary detection-reagents are well-known in the art and include chemiluminescent substrates, fluorescent substrates, and the like.

As used herein, the term “detecting” in the context of detecting the presence or amount of one or more analytes in a fluid sample refers to any method for determining the presence and/or amount of a particular analyte(s) present in the sample assayed. Detecting the presence of an analyte is qualitative in nature, such that no quantification of the analyte is required. Detecting the amount of analyte in a sample is quantitative, such that a relative amount of analyte is determined.

It is contemplated that virtually any assay, whether now known or developed in the future, may be employed to detect the analytes on the microplatform of the present device or in extracts taken from the microplatform of the present device. For instance, where the one or more analytes are detected on the microplatform, any binding assay that can be conducted on a membrane, including direct or sandwich assays may be employed. In one approach in such assays, analyte is bound to a binding member immobilized on the membrane, and a labeled second binding member is bound thereto to provide a detectable signal. The binding assays may be receptor-ligand assays including immunoassays, enzyme-substrate binding, and the like. It will also be appreciated that any variety of labels or indicator schemes, which provide a detectable signal that analyte binding has occurred, can be employed in the practice of the present invention. For example, direct labels such as fluorescent, radioactive and chromophoric labels can be used. Labels that may require development or enzymatic reagents, such as horseradish peroxidase or alkaline phosphatase, can also be utilized. It will likewise be appreciated that indirect label vehicles such as Protein A or avidin/biotin methods, know to those skilled in the art, can also be adapted for use with the device and assays of the present invention. In addition, electrochemical labels may be employed. Various multiplex detection techniques may be used such as, for example, the labeled bead set technology of U.S. Pat. No. 6,449,562 (Luminex), the relevant disclosure of which is incorporated herein by reference. Other examples of multiplexed detection include splitting the sample within the microdevice to be captured by separate microplatforms located within the microdevice; extraction of analytes from the microplatform and subsequent taking of aliquots of the extract for individual analysis of various analytes; using distinctive labels for each analyte within an analysis; having distinct capture loci for different analytes on a single microplatform; and so forth.

As mentioned above, analytes may be extracted from the microplatform and the extracts analyzed for the presence and/or amount of the one or more analytes. For example, the microdevices may be placed in respective wells of a solid surface structure comprising a plurality of receiving elements such as wells. The receiving element or the microdevice may be specifically designed to fit into a well of a standard microtiter plate such as, for example, a standard ELISA well. On the other hand, solid substrates may be designed with receiving elements that are based on the shape of the microdevice of the invention. A suitable extraction medium may be added to extract the analytes from the microplatform. Such extraction medium include, for example, an aqueous buffered medium at a moderate pH, generally that which provides optimum assay sensitivity. The aqueous medium may be solely water or may include from 0 to about 40 volume percent of a cosolvent, or may be a nonaqueous solvent for hydrophobic analytes (e.g., extraction of cyclosporine into methanol). The pH for the medium will usually be in the range of about 4 to about 11, more usually in the range of about 5 to about 10, and preferably in the range of about 6.5 to about 9.5. The extraction medium may include one or more detergents or surfactants. The pH, ionic strength, and the like of the medium are usually adjusted to provide maximum extraction of the analytes from the microplatform. The extraction medium may also suffice as the assay medium, which may require adjustment of the pH and the like.

Exemplary immunoassay formats contemplated herein, include the well-known sandwich assays, wherein the target-analyte is captured by an antibody, aptamer or receptor fixed to a microplatform and is detected either immediately, or later as described herein, using a labeled antibody, aptamer or receptor; competitive assays, wherein both labeled and unlabeled target-analyte compete for a fixed number of specific binding sites on antibodies, aptamers or receptors; sequential assay, wherein unlabeled analyte binds to a fixed number of binding sites, followed by a fill-in of the empty binding sites with labeled analyte either immediately or at a later time.

Another immunoassay contemplated herein, is a one-step immunoassay, wherein a labeled analyte (for example, latex, gold, carbon particles, or other agents visible without further processing) is used to prefill all available binding sites on an antibody capture-moiety. The capture antibody or other capture-moiety is selected so that it is easily competed off by unlabeled analyte during sample addition. Accordingly, the antibody for the target-analyte is selected so that the binding affinity between the antibody and the labeled analyte is weak enough for such competing off to occur to the extent necessary for the present assay. Such binding affinity is usually determined empirically. In place of selection of a weakly binding capture antibody, an analyte analog (chemically or otherwise altered analyte) may be selected to bind weekly to an existing antibody or other capture-moiety The remaining labeled analyte provides color that is inversely proportional to unlabeled analyte concentration.

Exemplary enzymatic assay formats contemplated herein includes an analyte first format, wherein the target-analyte is collected on a treated or untreated microplatform. An enzyme-reagent that is reactive with the target-analyte is added to the microplatform, either immediately, or at a later time as described herein, to quantify the analyte. Another enzymatic assay format is an enzyme first format wherein an enzyme for which the analyte is a substrate is first fixed to a microplatform material. Immediately upon addition of the sample containing the target-analyte, color development takes place, which can then be quantified either immediately or at a later time.

Exemplary receptor-ligand assays are well known in the art and include those described in, e.g., U.S. Pat. No. 5,856,092 (incorporated herein by reference in its entirety), and the like.

In certain embodiments, an advantage of the methods provided herein is that the detection step need not, and usually does not, occur immediately 1 5 after the target analyte binds to the microplatform. This advantage results from the stability of the target analyte on the microplatform. We have found that allowing the microplatform to dry attains such stability. This is usually accomplished by subjecting the microplatform to air drying at a temperature of about 15° C. to about 30° C., more usually, about 20° C. to about 25° C., preferably, ambient temperature, for a period of time sufficient for the microplatform to dry and become stabilized. Conveniently, this period of time may coincide with the time between collection of samples at one location and transport of the collected samples to another location for performing analysis such as performing a detection step. Drying may also be carried out under a gas other than air such as an inert gas, for example, nitrogen, noble gas, etc.

Accordingly, in certain embodiments, the drying time after fluid sample collection on the microplatform may be from about 2 hours to about 120 hours. In specific embodiments, the drying time is at least 2 hours, at least 3 hours, at least 4 hours, at least 6 hours, at least 8 hours, at least 10 hours, at least 12 hours, at least 15 hours, at least 20 hours, at least 24 hours, at least 48 hours, at least 72 hours, at least 96 hours, after the fluid sample is collected on the microplatform. For high throughput analysis involving multiple samples, multiple microplatforms and so forth, the drying time may be less than 2 hours, less than 1.5 hours, less than 1 hour, less than 0.5 hours, less than 0.1 hour, and the like.

This air drying may occur in a container that is open and in communication with the atmosphere or in a sealed vapor barrier container optionally with desiccant material such as alumina silica clays, silica gels, molecular sieves or the like (e.g., commercially available desiccants are available from Multisorb, Seneca, N.Y. or Sud-Chemie, Colton, N.Y., among others). The device itself may act as the vapor barrier, with desiccant material built into, for example, the cap of the device. Alternatively, the device or groups of devices may be sealed in a rigid or flexible vapor barrier container containing desiccants. After drying and during transport, certain analytes may require protection from ambient changes in humidity; the above described vapor barriers and desiccants would provide protection and permit shipment in a variety of ambient conditions. The aforementioned advantage applies to a capture moiety:analyte complex on the microplatform within the microdevice or the stability of the non-specifically captured analyte(s) on the microplatform.

The amount of desiccant is that sufficient to dry the sample such that the analyte is stable at ambient temperatures during shipping for periods of time from 1 day to 1 year. Desiccant material may be present in an amount of about 0.1 to about 5 grams, of about 0.5 to about 3 grams. Exemplary amounts of desiccant material are about 0.125 grams, about 0.5 grams, about 1 gram, about 1.5 grams, about 2 grams, about 2.5 grams, about 3 grams, about 3.5 grams, about 4 grams, about 4.5 grams, about 5 grams and amounts in between.

Accordingly, in certain embodiments, the detection step is conducted at a time after fluid sample collection of from about 2 hours to about year or more. In specific embodiments, the detection step is conducted at least 2 hours, at least 3 hours, at least 4 hours, at least 6 hours, at least 8 hours, at least 10 hours, at least 12 hours, at least 15 hours, at least 20 hours, at least 24 hours, at least 48 hours, at least 72 hours, at least 96 hours, at least 5 days, at least 6 days, at least 7 days, at least 2 weeks, at least 4 weeks, at least 6 weeks, at least 8 weeks, at least 10 weeks, at least 12 weeks, at least 15 weeks, at least 20 weeks, at least 25 weeks, at least 30 weeks, at least 35 weeks, at least 40 weeks, at least 45 weeks, at least 50 weeks, at least 55 weeks, or even, in certain embodiments, one year, two years, three years, five years, ten years, or decades, after the fluid sample is collected. For high throughput analysis involving multiple samples, multiple microplatforms and so forth, the detection step may be carried out less than 2 hours, less than 1.5 hours, less than 1 hour, less than 0.5 hours, less than 0.1 hour, and the like from the time of fluid sample collection.

In one embodiment, prior to detection, the step of organizing multiple microdevices having body fluid or non-body-fluid samples collected therein onto a solid-surface-structure is contemplated. This step of arranging the multiple microdevices onto a solid-surface (such as a microtiter plate, and the like) allows for high throughput analysis of the particular samples when used with conventional high throughput detection devices. Accordingly, also provided herein is a high throughput apparatus, comprising multiple microdevices spatially fixed to a solid-surface structure.

As use herein, the phrase “solid-surface-structure” refers to any surface onto or into which the microdevices may be arranged and organized, such as spatially, for subsequent analysis of the analyte to be detected. Any compatible surface can be used as a solid-surface in conjunction with the methods described herein. For example, solid-surfaces can be any of a variety of organic or inorganic materials or combinations thereof, including plastics, such as polypropylene or polystyrene; ceramic; silicon; (fused) silica, quartz or glass, which can have the thickness of, for example, a glass microscope slide or a glass cover slip. In a particular embodiment, the solid-surface is the plastic surface of a multiwell plate, for example a 24-, 96-, 256-, 384-, 864- or 1536-well plate.

The construction of the solid-surface can be varied such that it can be placed into any one of a variety of well-known detection (imaging) systems for automated detection (e.g., commercially available from numerous sources such as Molecular Devices, Sunnyvale, Calif.; Paul Bucher Company, Basel, Switzerland; and Alpha Innotech, San Leandro, Calif., and the like). Exemplary detection systems include, for example, the following systems: chemiluminescent (e.g., indirect detection using enzyme label and a chemiluminescent substrate; see, e.g., Bronstein et. al., 1989, Clin. Chem., 35:1441-1446); fluorescent (e.g., directly with fluorescent label or indirectly using an enzyme label and fluorogenic substrate; see, e.g., Johnson et al., 1986, Clin Chem, 32:378-381); calorimetric (e.g., directly using a colored label or indirectly using an enzyme label and a chromogenic substrate); and time-resolved fluorescence (e.g., directly using a fluorescent label; see, e.g., Barnard et al., 1998, Clin Chem, 44:1520-1528).

In one embodiment, the detection system is robotic. Exemplary robotic detection systems for use in the methods provided herein are well-known in the art and include, photometers (e.g., ELISA fluorescence, chemiluminescence, and absorbance microplate readers; reflectometers, and the like, which are commercially available from Molecular Devices, Sunnyvale, Calif.; and Paul Bucher Company, Basel, Switzerland), a CCD (charged coupled device; available from Alpha Innotech, San Leandro, Calif.), and the like.

As used herein, the phrase “organizing multiple microdevices” refers to the placement of multiple microdevices onto or into the solid-surface-structure (such as a plate) such that the multiple microdevices are organized into regions that are spatially discrete and addressable or identifiable. In certain embodiments, there are at least 2, 4, 6, 8, 10, 12, 15, 20, 24, 50, 96, 256, 384, 864, 1536, 2025, or more, spatially discrete (separated) regions containing microdevices. Increasing the number of regions on a solid-surface allows for assays of increasingly higher throughput. How the regions are separated, their physical characteristics, and their relative orientation to one another are not critical. In certain embodiments, the regions can be separated from one another by any physical barrier that is resistant to the passage of liquids. For example, the regions can be receiving elements such as wells of a multiwell dish or tissue culture plate (e.g., a 24-, 96-, 256-, 384-, 864- or 1536-well plate, or the like). Alternatively, a solid-surface such as a glass surface can be configured or etched out to have, for example, 864 or 1536 discrete, regions onto or into which the microdevices can be organized. In yet other embodiments, a solid-surface can comprise regions with no separations or wells, such as a flat surface (e.g., metal, a piece of plastic, glass, silicon, or paper), and individual regions can be spatially defined by overlaying a structure (e.g., a piece of plastic or glass), which delineates the separate regions.

Accordingly, also provided herein are high throughput methods of detecting one or more analytes in a fluid sample, comprising:

a) collecting one or more microvolume samples of fluid directly into one or more microdevices;

b) transporting each fluid sample within the microdevice to a particular locus on a microplatform housed within the device, wherein said locus optionally contains a capture-moiety specific for said analyte;

c) organizing multiple microdevices having fluid samples collected therein, onto a solid-surface; and

d) detecting the presence or amount of one or more analytes in said fluid samples.

Also provided herein are high throughput methods of detecting one or more analytes within whole blood of multiple subjects undergoing clinical trials, comprising:

a) collecting one or more microvolume samples of whole-blood from a subject directly into one or more microdevices;

b) transporting each whole-blood sample within the microdevice through a cell separating mechanism, so that blood serum is delivered to a particular locus on a microplatform housed within the device, wherein said locus optionally contains a capture-moiety specific for said analytes;

c) organizing multiple microdevices having blood samples collected therein, onto a solid-surface; and

d) detecting the presence or amount of one or more analytes in said blood samples.

The present devices and methods may be applied to any test requiring a blood sample, including pediatric clinical trials and toxicology or other studies in laboratory animals, companion animals, food animals, or wild animals with a capability of analyzing one or more substances simultaneously in a drop of blood. By allowing the collection and high throughput analysis of virtually any analyte in very small samples, it permits scientific protocols currently impossible or extremely difficult, such as serial blood sampling from a single mouse. The number of substances may be from one to about 100 or more substances. The present invention simplifies the pre-analytical processes of sample collection and sample preparation, which are the most time-consuming and expensive parts of biological testing and during which most common mistakes occur. By automating these processes, the present invention significantly reduces the pre-analytical error rate, improves test quality, substantially reduces labor costs, and reduces discomfort from sample collection. The present devices and methods may be used with existing testing instrument systems.

Various high throughput logistical formats are contemplated herein for employing the methods and devices provided herein, such as a laboratory format, an onsite format, and the like. In the laboratory format, for example, the microdevices are packaged in organizing racks (also referred to herein as solid-surface-structures) and shipped to clinical sites. Prior to shipping to clinical sites, the microdevices devices are fixed or locked in place (e.g. threaded into a proprietary holder), or held in place by some other mechanical means (e.g., a tightly fitting cover). Once the fluid is collected into the microdevice, the microdevice is reloaded into the racks (such as an 8 cm×12 cm rack equal in size to an ELISA plate) and securely fixed in place. The patient and sample information is recorded on each device, and the racks are shipped to an analytical laboratory or tested at an onsite laboratory. At the laboratory, the loaded racks are run either manually or on automated ELISA equipment.

Another logistical assay format contemplated herein is the onsite where the microdevice is packaged in organizing racks and shipped to clinical sites. As described above, the microdevice is locked or covered in place. The patient and sample information is recorded on each device. Either immediately, hours, days, weeks or months after sample collection, the sample is placed into detection device. The detection device reads an amount of analyte present using detection-reagents in bulk in the microdevice; the detection-reagents unit packaged in each collection microdevice; using no reagents needed for the “enzyme first” format; or no reagents needed for the “one step” format. The microdevice stores patient information, sample information and sample result for display, analysis, downloading and/or transmission to a remote location or separate device.

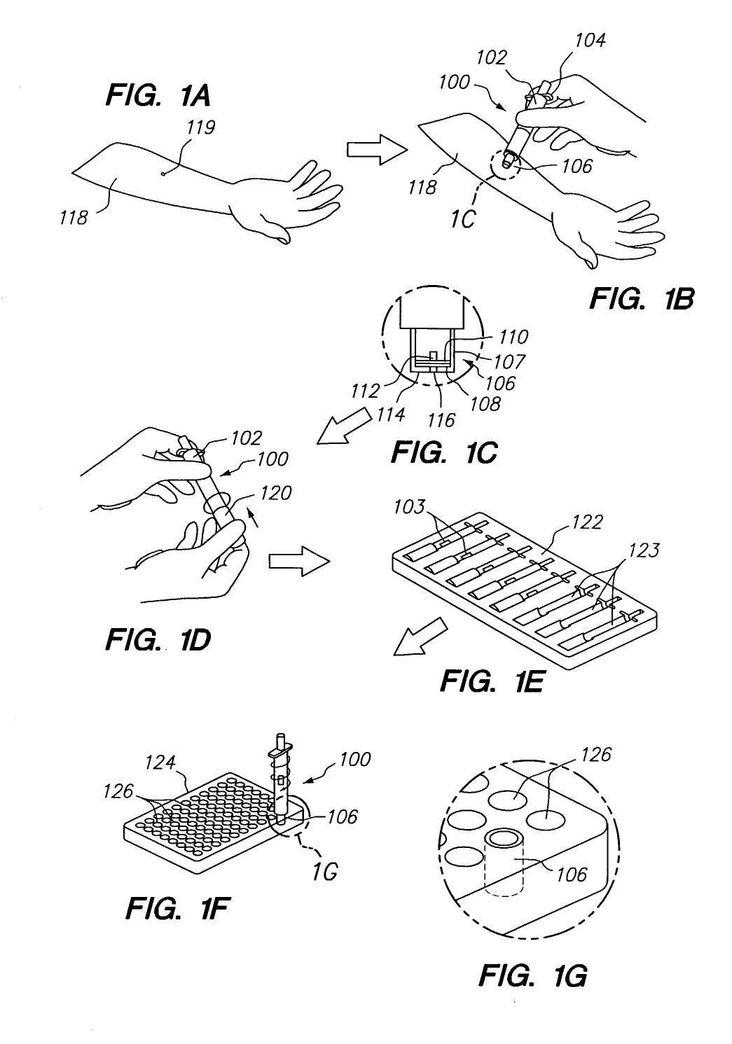

An example of a device in accordance with the present invention is depicted in FIGS. 1B-1G. Device 100 comprises main body 102 with bar code 103, flared top portion 104 and assay module 106, which is detachably secured to the base of main body 102 by means of, for example, a screw mechanism, friction fit, and the like. Bar code 103 may include identifying indicia such as patient information, date of collection, and so forth. Assay module 106 comprises housing 107 in which filter 108, membrane (microplatform) 110 with membrane tab 112 are seated. Bottom wall 114 of housing 107 has opening 116, which is designed to permit a predetermined volume of sample to be absorbed into assay module 106. In one approach, the size of opening 116 and the absorption volume capacity of membrane 110 are chosen to take in a predetermined volume of sample into assay module 106.

In another approach, particularly for plasma, membrane 110 is situated such that it is in constant contact with filter 108. Whole blood is drawn in through 116 and plasma is separated out by filter 108. The plasma is immediately drawn into membrane 110. Whole blood is continually added through 116 until membrane 110 has absorbed a predetermined volume of sample, as indicated by tab 112. In another approach, particularly for plasma, the absorption capacity of membrane 110 only governs the predetermined volume of sample.

In another approach, particularly for whole blood, the amount of whole blood collected is governed by the absorption capacity of filter 108. In this approach, tab 112 is in contact with filter 108 to assist in determining when the filter is full. Alternatively housing 114 is clear, allowing filter 108 to be observed directly. When filter 108 is full it is brought in contact with membrane 110 by replacing cap 120 or the like. The filter-membrane contact may occur by means of a small pin in cap 120 that fits through opening 116, which impinges on filter 108, bowing the filter to come in contact with membrane 110 at a single point. The absorption capacity of membrane 110 then collects a predetermined volume of plasma from the blood separation filter 108.

In another approach a short fill tube may be employed that is then brought into contact with a blood separation membrane after the fill tube is full of fluid. This approach is useful for metering whole blood. Thus, as an example, bottom wall 114 of device 100 is gently touched to a patient's member such as arm 118, which has been pierced by a pinprick to form a drop of blood 119 on arm 118 (FIG. 1A). The blood sample travels through opening 116 and through filter 108, which separates plasma from the whole blood. The plasma travels into membrane 110, which may capture one or more analytes in the sample by specific capture agents on the membrane or may simply absorb the analytes in some non-specific manner. Membrane tab 112 is designed to change color when the predetermined volume of sample has been absorbed on membrane 110. In one approach, such color change may be realized by employing, for example, pH indicator paper selected in a broad pH range to correspond to the intended sample.

In another approach, membranes or filters may be used having a soluble dye such as bromophenol blue applied to the end of membrane tab 112 that is in contact with membrane 110. In yet another approach, moisture indicator paper may be employed. The moisture indicator paper is designed to respond to humidity levels of 95% (commercially available from Sud-Chemie (Colton, Calif.)), sealed from atmospheric moisture by lamination, and to respond solely to wicked moisture from membrane 110 and the like.

After collection of the predetermined volume of sample into assay module 106, cap 120 is secured to device 100 by means of a screw mechanism, a friction fit, and so forth. Device 100 is then placed in tray 122, which comprises a plurality of recesses 123 designed to receive a plurality of devices 100. Device 100 with cap 120 in place is designed to provide adequate drying of the sample on membrane 110. Desiccant material may be included in cap 120 alone, or the cap and the body of device 100.

Tray 122 is transported to a laboratory, such as a registered commercial medical diagnostic laboratory, a research laboratory, an on-site laboratory, a laboratory operated by the manufacturer of the device or the like where the samples are to be analyzed. Each device 100 is removed from tray 122 and cap 120 is removed and discarded. Cap 120 is designed to remove filter 108. To this end cap 120 may be designed to snap on during manufacture, such that cap removal does not remove filter 108, and to screw on after sample collection, such that the attachment at this post sample collection stage engages a portion of the device that secures filter 108 and removes filter 108 when the cap is next removed.

Each device 100 is moved to block 124 comprising a plurality of wells 126, which are designed to receive assay module 106. Conveniently, block 124 may be a microtiter plate and the shape of assay module 106 generally conforms to the shape of a well in the microtiter plate. Assay module 106 of device 100 is placed into one of wells 126 so that membrane 110 is seated at the bottom of well 126. Once a desired number of assay modules 106 are seated in wells 126, an assay is carried out to determine the presence and/or amount of one or more analytes that may have been present in the samples from the patient. Any convenient assay may be employed such as, for example, those assays described hereinabove. The present devices provide for a simple and convenient way to perform high throughput analysis on samples to be analyzed. For example, a patient may be monitored over a period of time by taking samples from the patient at predetermined intervals using the devices of the invention. After an appropriate number of samples have been taken, they can be transported to a location for analysis. The samples are dried on the membrane of the present device and are stable over extended periods of time for subsequent analysis. Main body 102 and cap 120 may be fabricated from any suitable material that provides the necessary structural strength and optionally acts as a vapor barrier for the above articles. Such materials include, for example, plastic, lightweight composites, and the like.

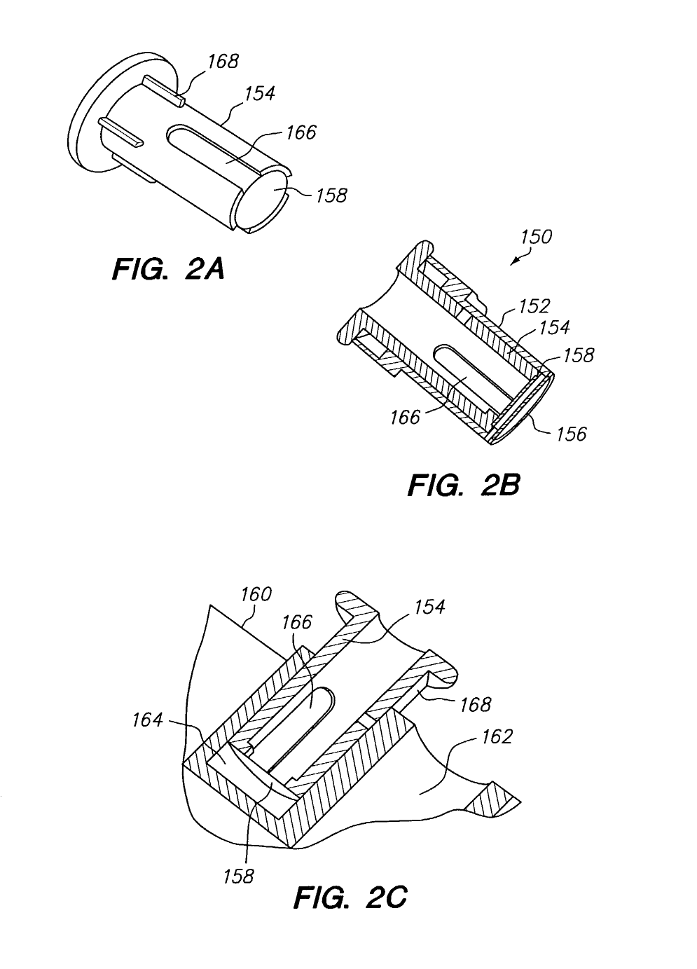

Another example of an assay module is depicted in FIGS. 2A-2C. Assay module 150 is depicted and comprises outer housing 152 and inner housing 154. Outer housing 152 comprises filter 156 and inner housing 154 comprises membrane 158. Sample such as whole blood is drawn into assay module 150 at filter 156, which is designed to absorb a predetermined volume of sample. Optionally, the blood could be brought directly in contact with membrane 158. In the former case, thereafter, filter 156 is urged into contact with membrane 158 by any suitable means such as, for example, a probe tip pushing from the side of membrane 158 toward filter 156, or in the reverse direction. The urging means may be a part of a cap for covering assay module 150, part of a separate plate with one or more of such probes, a slotted plate that presses a plunger against membrane 158 when device 150 is placed into it, a weighted device that presses a raised area against membrane 158 with a constant pressure and so forth. When the sample on membrane 158 is to be analyzed, a cap is removed, thereby removing outer housing 152 from inner housing 154, which is inserted into block 160 comprising a plurality of recesses 162 designed to receive, seat and hold housing 154. Then, the sample from membrane 158 is analyzed to determine the presence and/or amount of one or more analytes on the membrane. In the example shown in FIG. 2C, housing 154 seats in recess 162 so that chamber 164 is formed. In this example, analyte is extracted with a suitable extraction fluid from membrane 158 into chamber 164 and a suitable assay is performed on the extract. As can be seen from FIGS. 2A-2C, inner housing 154 comprises slots 166 to allow free flow of extraction fluid and ridges 168 to set placement of inner housing 154 in recess 162 of a block 160.

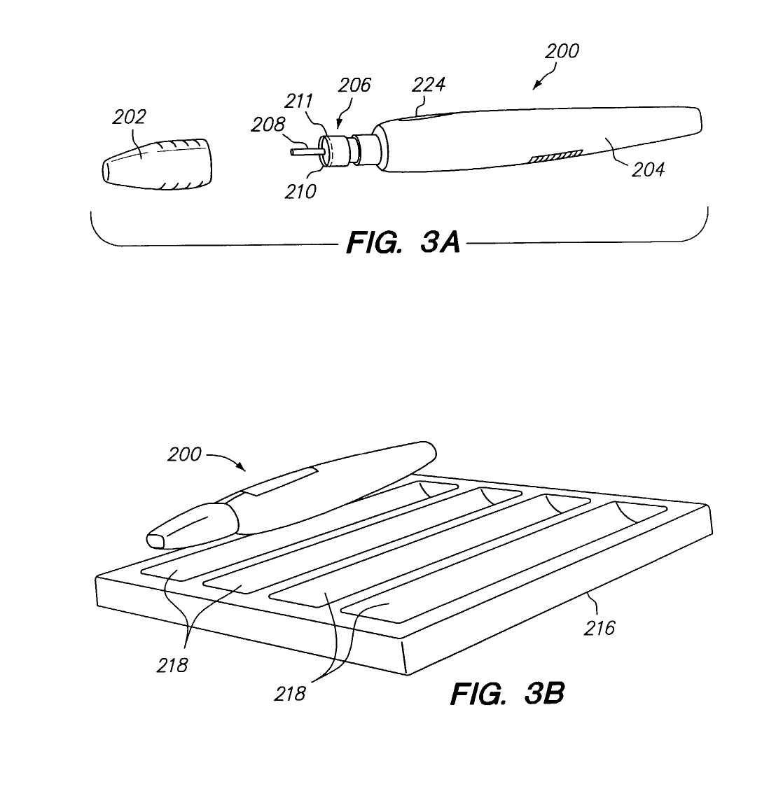

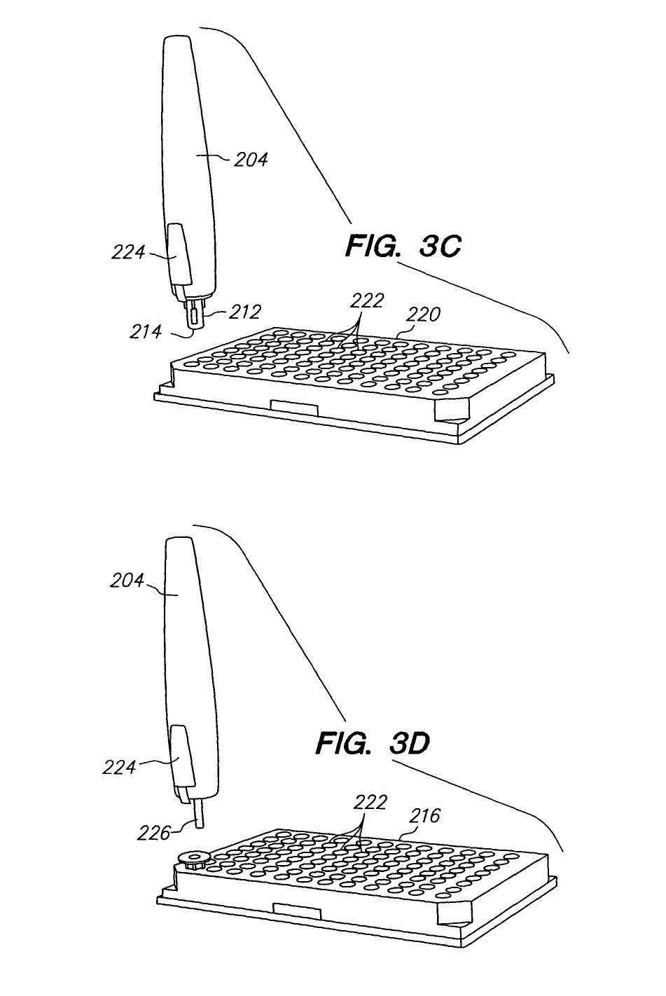

Another embodiment of a device in accordance with the present invention is depicted in FIGS. 3A-3D. Device 200 comprises cap 202, main body 204 and assay module 206. In the embodiment shown, assay module 206 comprises capillary tube 208, filter assembly 210 and housing 212 with microplatform or membrane 214. Device 200 also comprises release button 224, which is designed to release housing 212 as explained below. In operation, cap 202 is removed from main body 204 thereby exposing capillary tube 208. Device 200 is manipulated to bring capillary tube 208 into fluid communication with a sample such as a whole blood sample. Sample is drawn into capillary tube 208 by capillary action and is absorbed by filter 211 of filter assembly 210. In certain embodiments capillary tube 208 may comprise a suitable tip for penetrating the skin of a patient that may be employed for generating a sample from the patient. When a predetermined volume of sample is absorbed by filter 211, a color change occurs on filter 211. Such color change may be achieved by means such as described above. Alternatively, for collection of colored liquids such as whole blood, the filter 211 will take up the color of the fluid as it absorbs it, thereby indicting when filter 211 has become saturated. Filter assembly 210 is conveniently fabricated from a transparent material, e.g., transparent plastic, glass, and the like, so that such color formation or changes may be visualized. Cap 202 is secured to main body 204 and urges capillary tube 208, which in turn urges filter 211 into contact with membrane 214 resulting in the transfer of a predetermined volume of sample (plasma in the case of whole blood) to membrane 214. Device 200 is placed into tray 216, which comprises a plurality or recesses 218 having a shape that generally corresponds with the shape of device 200. It should be noted that the shape of the recesses need not conform exactly to that of device 200. The shape of the recesses may be any convenient form with appropriate ridges and the like to provide relative immobility to device 200 within recess 218.

Tray 216 is transported to a laboratory, such as a registered commercial medical diagnostic laboratory, a research laboratory, an on-site laboratory, a laboratory operated by the manufacturer of the device or the like where the samples are to be analyzed. Each device 200 is removed from tray 216 and cap 202 is removed and discarded. Cap 202 is designed to remove capillary tube 208 and filter assembly 210, which are discarded along with cap 202. Each device 200 is moved to block 220 comprising a plurality of wells 222, which are designed to receive housing 212. Housing 212 of device 200 is placed into one of wells 222 so that membrane 214 is seated in well 222. Release button 224 is activated to release housing 212 from spindle 226 and main body 204 is withdrawn leaving housing 212 in well 222. As can be seen, main body 204 comprises spindle 226, on which housing 212 was removably secured by, for example, a small clip at the end of button 224, and the like. Once a desired number of housings 212 are seated in wells 222, assays are carried out to determine the presence and/or amount of one or more analytes that may have been present in the samples from the patient.

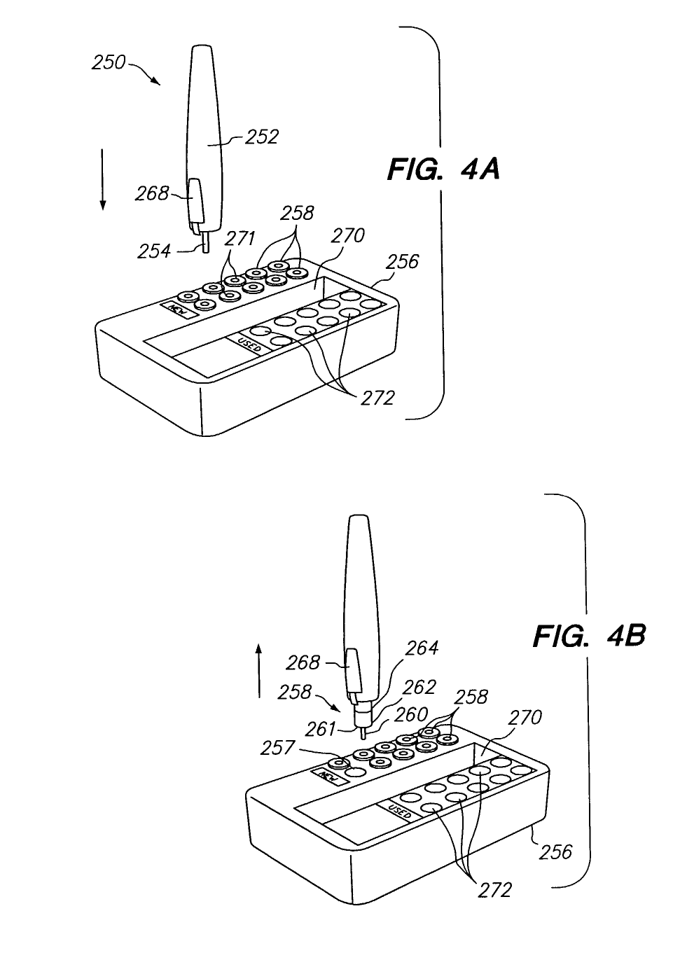

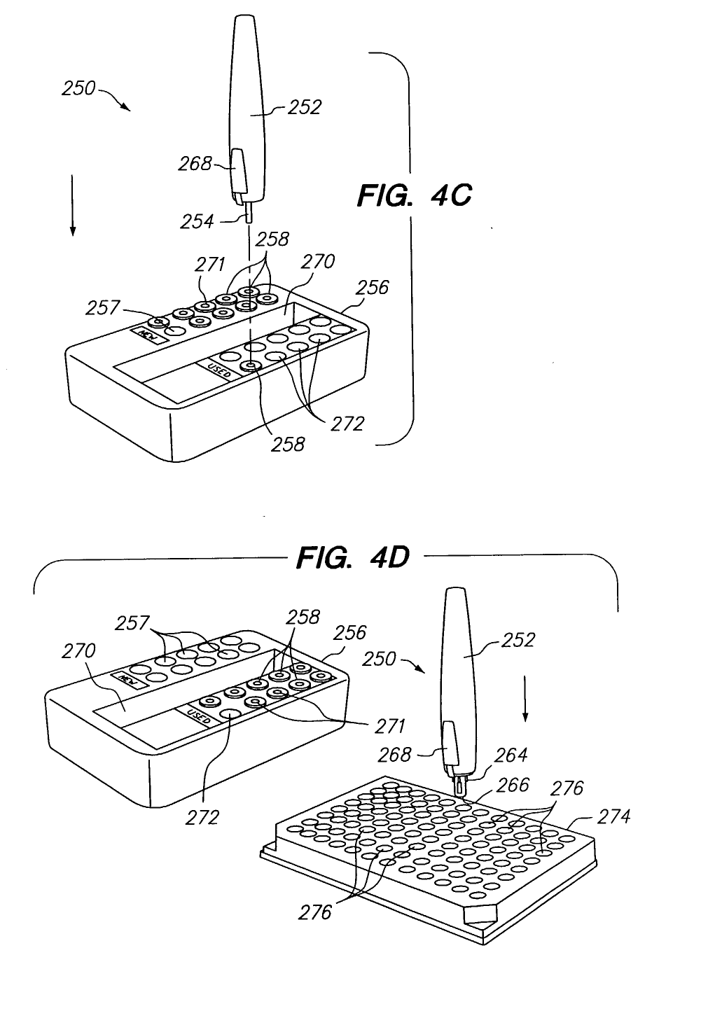

Another embodiment of a device in accordance with the present invention is depicted in FIGS. 4A-4D. Device 250 comprises main body 252 and spindle 254. Tray 256 comprises a plurality of wells 257, each containing an assay module 258. In the embodiment shown, assay module 258 is similar to assay module 206 described above and comprises capillary tube 260, filter assembly 262 and housing 264 with microplatform or membrane 266. Device 250 also comprises release button 268, which is designed to release housing 264 as explained above for device 200. Tray 256 also comprises recess 270 20 for storing device 250. Tray 256 also comprises a plurality of wells 272 designed to receive assay module 258.

In operation, device 250 is removed from recess 270 and moved in a direction toward an assay module 258 seated in well 257. Spindle 254 enters bore 271 in assay module 258, which becomes releasably secured thereto. Any convenient mechanism may be employed for releasably securing assay module 258 to spindle 254 including, for example, a small clip on the end of the lever-actioned button, 268, which is spring loaded and secures assay module 258 by means of a small lip to device 252, and the like. Device 250 with assay module 258 attached is then used as described above for device 200. To this end, device 250 is manipulated to bring capillary tube 260 into fluid communication with a sample such as a whole blood sample. Sample is drawn into capillary tube 260 by capillary action and is absorbed by filter 261 of filter assembly 262. When a predetermined volume of sample is absorbed by filter 261, a color change occurs on filter 261. Such color change may be achieved by a means such as described above. Device 250 is then moved to one of wells 272 where assay module 258 is inserted. Release button 268 is activated to release assay module 258 from spindle 254 and device 250 is withdrawn leaving assay module 258 in well 272. The placement of assay module 258 in well 272 urges capillary tube 260, which in turn urges filter 261 into contact with membrane 266 resulting in the transfer of a predetermined volume of sample (plasma in the case of whole blood) to membrane 266.

When the desired number of samples have been collected, device 250 is placed in recess 270 and tray 256 is hermetically sealed with a suitable cover, optionally containing desiccants (not shown) and is transported to a laboratory, such as a registered commercial medical diagnostic laboratory, a research laboratory, an on-site laboratory, a laboratory operated by the manufacturer of the device or the like where the samples are to be analyzed. Device 250 is removed from tray 256 and spindle 254 is inserted into bore 271 of assay module 258 and releasably secures housing 264. Wells 272 are designed such that capillary tube 208 and filter assembly 210 are removed from housing 264 and retained in wells 272. Such design may be, for example, a one-way securing mechanism in which the depth of well 272 is such that the perimeters of capillary tube 208 and filter assembly 210 fit into a one-way friction snap fitting that secures those components to tray 256. Other approaches will be evident to those skilled in the art in view of the above. Each device 250 is moved to block 274 comprising a plurality of wells 276, which are designed to receive housing 264 as described above in FIGS. 3A-3D. Housing 264 is placed into one of wells 276 so that membrane 266 is seated in well 276. Release button 268 is activated to release housing 264 from spindle 254 and main body 252 is withdrawn leaving housing 264 in well 276. Once a desired number of housings 264 are seated in wells 276, assays 30 are carried out to determine the presence and/or amount of one or more analytes that may have been present in the samples from the patient.

Specific Embodiments of the Invention

Provided herein are methods for detecting one or more analytes in a fluid sample, comprising:

a) collecting one or more microvolume samples of fluid directly into one or more microdevices;

b) transporting each fluid sample within the microdevice to a particular locus on a microplatform housed within the device, wherein said locus contains a capture-moiety specific for each of said one or more analytes; and

c) detecting the presence or amount of one or more analytes in said fluid samples.

Provided herein are methods for detecting one or more analytes in a fluid sample, comprising:

a) collecting one or more microvolume samples of fluid directly into one or more microdevices;

b) transporting each fluid sample within the microdevice to a particular locus on a microplatform housed within the device, wherein said locus optionally contains a capture-moiety specific for each of said one or more analytes; and

c) detecting the presence or amount of one or more analytes in said fluid samples.

Also provided herein are high throughput methods of detecting one or more analytes in a fluid sample, comprising:

a) collecting one or more microvolume samples of fluid directly into one or more microdevices;

b) transporting each fluid sample within the microdevice to a particular locus on a microplatform housed within the device, wherein said locus optionally contains a capture-moiety specific for said analyte;

c) organizing multiple microdevices having fluid samples collected therein, onto a solid-surface; and

d) detecting the presence or amount of one or more analytes in said fluid samples.

Further provided herein are high throughput methods of detecting one or more analytes within whole blood of multiple subjects undergoing clinical trials, comprising:

a) collecting one or more microvolume samples of whole-blood from a subject directly into one or more microdevices;

b) transporting each whole-blood sample within the microdevice through a cell separating mechanism, so that blood plasma is delivered to a particular locus on a microplatform housed within the device, wherein said locus optionally contains a capture-moiety specific for said analytes;

c) organizing multiple microdevices having blood samples collected therein, onto a solid-surface; and

d) detecting the presence or amount of one or more analytes in said blood samples.

Also provided herein are microdevices, comprising a mechanism for collecting and transporting a fluid sample within the microdevice; a microplatform; a biochemical capture-moiety attached to the microplatform; and a housing in which each of these components resides. In another embodiment the microdevices comprise a mechanism for collecting a fluid sample into the microdevice, a mechanism for transporting a fluid sample within the microdevice; a microplatform; a biochemical capture-moiety attached to the microplatform; and a housing in which each of these components resides. The microdevices can further comprise, when said fluid sample is a body-fluid sample, a cell separating mechanism so that blood serum is delivered to a particular locus on the microplatform housed within the device. In addition, the microdevice provided herein can further comprise a harvesting mechanism for making the body-fluid-sample available for collection, wherein said harvesting mechanism is integrated into the microdevice, such that the harvesting and subsequent collection of the body-fluid sample into the microdevice is accomplished using a single structure.

Also provided herein is an apparatus for high throughput fluid analysis, such as body and non-body fluid analysis, comprising multiple microdevices, spatially fixed to a solid-surface structure.

These methods and devices provided herein are useful for qualitatively detecting the presence of, and/or quantitatively determining the amount of, one or more analytes in a relatively small microvolume fluid sample, including body-fluid samples and non-body fluid samples. In particular, the methods and devices provided herein are useful for high throughput detection of body-fluid analytes that are analyzed in bulk, such as during clinical trials or the like. The methods and devices provided herein are also useful for determining the metabolic and physiologic phenotypes and genotypes or genetic material analysis of animals including humans, research animals such as transgenic animals, such as when only minute microvolumes of body fluid are available. Genotype determination may be from red cells or other cells found in a body fluid such as, for example, an extract from a tumor. The methods and devices provided herein are also useful for the high or low throughput detection of analytes in a non-body fluid sample, such as tissue culture media, dialysate, and the like. In addition, the methods and devices are useful for detecting analytes in fluid samples that are collected at one location, such as a clinic or doctors office for diagnostic purposes, and shipped to a separate location for detection analysis.

Also provided are methods for detecting one or more analytes in a fluid sample. A predetermined microvolume of the fluid sample is collected into a microdevice, preferably, directly collected into a microdevice. The one or more analytes of the fluid sample are transported within the microdevice to a microplatform housed within the device. The microplatform comprises a capture moiety for each of the one or more analytes. The capture moiety may capture the one or more analytes specifically or non-specifically. In this way the fluid sample is subjected to a metering function within the present apparatus. The presence or amount of the one or more analytes is detected either on or off the microplatform. The presence or amount of these analytes detected indicates the presence or amount of the one or more analytes in the fluid sample.

Also provided are methods for detecting one or more analytes in a body-fluid sample, comprising:

a) collecting one or more microvolume samples of body-fluid from a subject directly into one or more microdevices, wherein each body-fluid sample is transported within the microdevice to a particular locus on a microplatform housed within the device, wherein said locus contains a capture-moiety specific for each of said one or more analytes; and

b) detecting the presence or amount of one or more analytes in said body-fluid samples. One specific embodiment is a method for detecting one or more analytes in a fluid sample, comprising:

a) collecting one or more microvolume samples of fluid directly into one or more microdevices;

b) transporting each fluid sample within the microdevice to a particular locus on a microplatform housed within the device, wherein the locus contains a capture-moiety specific for each of the one or more analytes; and

c) detecting the presence or amount of one or more analytes in the fluid samples.

The method may further comprise prior to detection step (c), organizing multiple microdevices having fluid samples collected therein, onto a solid-surface.

The fluid sample may be a body-fluid sample obtained from a subject, wherein the body-fluid sample is selected from the group consisting of whole-blood, plasma, serum, interstitial fluid, sweat, saliva, urine, semen, blister fluid, inflammatory exudate, body-gas and body-vapor.

The body-fluid sample may be whole blood, and the whole-blood sample is transported through a cell separating mechanism so that blood serum is delivered to the particular locus on the microplatform housed within the device.

In the above method the body-fluid sample may be collected by a collection-means selected from the group consisting of: a lancet, a microneedle, and skin ablation.

In the above method the microplatform may comprise a material selected from the group consisting of a membrane, a filter, a plastic support, a silicon support, a glass support.

In the above method the locus may be planar ranging in size from no greater than 3000, 2500, 2000, 1500, 1000, 500, 400, 300, 200, 100, 75, 50, 40, 30, 25, 20, 15, 10, 5, 4, 3, 2, 1, 0.5, 0.1, 0.01, 0.001 mm²; or is volumetric ranging in volume from 1 nl to 250 microliters.

In the above method the capture-moiety may be selected from the group consisting of an anti-analyte-specific antibody and an anti-analyte-specific aptamer, and an anti-analyte-specific reactive-enzyme.

In the above method the detection step (c) may be carried out by an assay selected from the group consisting of an immunoassay, an enzyme assay, a receptor-ligand assay, and an electrochemical assay.

In the above method the solid-surface may be placed into an automated detection system.

In the above method the solid-surface may contain a multiple of microdevices organized thereon selected from the group consisting of: at least 6; at least 12; at least 24; at least 48; at least 96; at least 256; at least 15 384; at least 864; at least 1536.

In the above method the analytes may be detected by adding a detection-reagent to the microplatform.

In the above method the detection-reagent may not move laterally through the microplatform.

In the above method the detection-reagent may be added to the platform at a different geographic location than the location of sample collection.

In the above method the detection step may be conducted at a time after fluid sample collection selected from the group consisting of: at least 30 minutes, at least one hour, at least 2 hours, at least 4 hours, at least 6 hours, at least 8 hours, at least 10 hours, at least 12 hours, at least 15 hours, at least 20 hours, at least 24 hours, at least 48 hours, at least 72 hours, at least 96 hours, at least 5 days, at least 6 days, at least 7 days, at least 2 weeks, at least 4 weeks, at least 6 weeks, at least 8 weeks, at least 10 weeks, at least 12 weeks, at least 15 weeks, at least 20 weeks, at least 25 weeks, at least 30 weeks, at least 35 weeks, at least 40 weeks, at least 45 weeks, at least 50 weeks, at least 55 weeks.

In the above method the microvolume may be selected from the group consisting of: less than 250 microliters, 200 microliters, 150 microliters, 100 microliters, 50 microliters; 40 microliters; 30 microliters; 20 microliters; 10 microliters; 5 microliters; 1 microliter; 900 nl; 800 nl; 700 nl; 600 nl; 500 nl; 400 nl; 300 nl; 200 nl; 100 nl; 75 nl; 50 nl; 40 nl; 30 nl; 20 nl; 15 nl; 10 or 1 nl.

In the above method the microplatform may be a membrane.

In the above method the locus on the membrane may be no greater than a 40 mm².

In the above method the body-fluid sample may be harvested using a lancet.

In the above method the transporting step (b) may be by capillary means.

Another specific embodiment of the invention is a high throughput method of detecting one or more analytes within whole blood of multiple subjects undergoing clinical trials, comprising:

a) collecting one or more microvolume samples of whole-blood from a subject directly into one or more microdevices;

b) transporting each whole-blood sample within the microdevice through a cell separating mechanism, so that blood serum is delivered to a particular locus on a microplatform housed within the device, wherein the locus contains a capture-moiety specific for the analytes;

c) organizing multiple microdevices having blood samples collected therein, onto a solid-surface; and

d) detecting the presence or amount of one or more analytes in the blood samples.

In the above method the solid-surface may be placed into a detection system prior to detection step (d).

In the above method the detection system may be robotic.

In the above method the whole blood sample may be collected by a collection-means selected from the group consisting of: a lancet, a microneedle, and skin ablation.

In the above method the microplatform may comprise a material selected from the group consisting of a membrane, a filter, a plastic support, a silicon support, a glass support.

In the above method the locus may be planar ranging in size from 3000, 2500, 2000, 1500, 1000, 500, 400, 300, 200, 100, 75, 50, 40, 30, 25, 20, 15, 10, 5, 4, 3, 2, 1, 0.5, 0.1, 0.01, 0.001 mm²; or is volumetric ranging in volume from 1 nl to 100 microliters.

In the above method the capture-moiety may be selected from the group consisting of an anti-analyte-specific antibody and an anti-analyte-specific aptamer, and an anti-analyte-specific reactive-enzyme.

In the above method the detection step (c) may be carried out by an assay selected from the group consisting of an immunoassay, an enzyme assay, a receptor-ligand assay, and an electrochemical assay.

In the above method the solid-surface may contain a multiple of microdevices organized thereon selected from the group consisting of: at least 6; at least 12; at least 24; at least 48; at least 96; at least 256; at least 384; at least 864; at least 1536.

In the above method the analytes may be detected by adding a detection-reagent to the microplatform.

In the above method the detection-reagent may not move laterally through the microplatform.

In the above method the detection-reagent may be added to the platform at a different geographic location than the location of sample collection.

In the above method the detection step may be conducted at a time after whole-blood sample collection selected from the group consisting of: at least 30 minutes, at least one hour, at least 2 hours, at least 4 hours, at least 6 hours, at least 8 hours, at least 10 hours, at least 12 hours, at least 15 hours, at least 20 hours, at least 24 hours, at least 48 hours, at least 72 hours, at least 96 hours, at least 5 days, at least 6 days, at least 7 days, at least 2 weeks, at least 4 weeks, at least 6 weeks, at least 8 weeks, at least 10 weeks, at least 12 weeks, at least 15 weeks, at least 20 weeks, at least 25 weeks, at least 30 weeks, at least 35 weeks, at least 40 weeks, at least 45 weeks, at least 50 weeks, at least 55 weeks.

In the above method the microvolume may be a quantity selected from the group consisting of: less than 250 microliters, 200 microliters, 150 microliters, 100 microliters, 50 microliters; 40 microliters; 30 microliters; 20 microliters; 10 microliters; 5 microliters; 1 microliter; 900 nl; 800 nl; 700 nl; 600 nl; 500 nl; 400 nl; 300 nl; 200 nl; 100 nl; 75 nl; 50 nl; 40 nl; 30 nl; 20 nl; 15 nl; 10 nl; 5 nl or 1 nl.

In the above method the microplatform may be a membrane.

In the above method the locus on the membrane may be no greater than a 40 mm².

In the above method the whole blood sample may be harvested by a lancet.

In the above method the transporting step (b) may be by capillary action.

Another embodiment of the present invention is a microdevice, comprising a mechanism for collecting and transporting a fluid sample within the microdevice; a microplatform; a biochemical capture-moiety attached to the microplatform; and a housing in which each of these components resides.

Another specific embodiment of the present invention is a microdevice, comprising a mechanism for collecting a fluid sample into the microdevice, a mechanism for transporting a fluid sample within the microdevice; a microplatform; a biochemical capture-moiety attached to the microplatform; and a housing in which each of these components resides.

In the above microdevices, when the fluid sample is a body-fluid sample, the microdevice may further comprise a cell separating mechanism so that blood serum is delivered to a particular locus on the microplatform housed within the device.

The microdevice may further comprise a harvesting mechanism for making the body-fluid-sample available for collection, wherein the harvesting mechanism is integrated into the microdevice, such that the harvesting and subsequent collection of the body-fluid sample into the microdevice is accomplished using a single structure.

Another specific embodiment of the invention is an apparatus for high throughput body-fluid analysis, comprising multiple microdevices as described above.

Another specific embodiment of the invention is a high throughput method of detecting one or more analytes in a fluid sample, comprising:

a) collecting one or more microvolume samples of fluid directly into one or more microdevices;

b) transporting each fluid sample within the microdevice to a particular locus on a microplatform housed within the device, wherein the locus contains a capture-moiety specific for the analyte;

c) organizing multiple microdevices having fluid samples collected therein, onto a solid-surface; and

d) detecting the presence or amount of one or more analytes in the fluid samples.

In the above method the solid-surface may be placed into a detection system prior to detection step (d).

In the above method the detection system may be robotic.

In the above method the microplatform may comprise a material selected from the group consisting of a membrane, a filter, a plastic support, a silicon support, a glass support.

In the above method the locus may be planar ranging in size from 3000, 2500, 2000, 1500, 1000, 500, 400, 300, 200, 100, 75, 50, 40, 30, 25, 20, 15, 10, 5, 4, 3, 2, 1, 0.5, 0.1, 0.01, 0.001 mm²; or is volumetric ranging in volume from 1 nl to 100 microliters.

In the above method the capture-moiety may be selected from the group consisting of an anti-analyte-specific antibody and an anti-analyte-specific aptamer, and an anti-analyte-specific reactive-enzyme.

In the above method the detection step (c) may be carried out by an assay selected from the group consisting of an immunoassay, an enzyme assay, a receptor-ligand assay, and an electro-chemical assay.

In the above method the solid-surface may contain a multiple of microdevices organized thereon selected from the group consisting of: at least 6; at least 12; at least 24; at least 48; at least 96; at least 256; at least 384; at least 864; at least 1536.

In the above method the analytes may be detected by adding a detection-reagent to the microplatform.

In the above method the detection-reagent may be added to the platform at a different geographic location than the location of sample collection.

In the above method the detection step may be conducted at a time after sample collection selected from the group consisting of: at least 30 minutes, at least one hour, at least 2 hours, at least 4 hours, at least 6 hours, at least 8 hours, at least 10 hours, at least 12 hours, at least 15 hours, at least 20 hours, at least 24 hours, at least 48 hours, at least 72 hours, at least 96 hours, at least 5 days, at least 6 days, at least 7 days, at least 2 weeks, at least 4 weeks, at least 6 weeks, at least 8 weeks, at least 10 weeks, at least 12 weeks, at least 15 weeks, at least 20 weeks, at least 25 weeks, at least 30 weeks, at least 35 weeks, at least 40 weeks, at least 45 weeks, at least 50 weeks, at least 55 weeks.

In the above method the microvolume may be a quantity selected from the group consisting of: less than 250 microliters, 200 microliters, 150 microliters, 100 microliters, 50 microliters; 40 microliters; 30 microliters; 20 microliters; 10 microliters; 5 microliters; 1 microliter; 900 nl; 800 nl; 700 nl; 600 nl; 500 nl; 400 nl; 300 nl; 200 nl; 100 nl; 75 nl; 50 nl; 40 nl; 30 nl; 20 nl; 15 nl; 10 nl; 5 nl or 1 nl.

Results from the detection step may be raw results (such as fluorescence intensity readings and the like) or may be processed results such as data obtained by some analysis of the results, either manually or by computer and/or forming conclusions based on the analysis. The results (processed or not) may be forwarded (such as by communication) to a remote location if desired, and received there for further use (such as further processing or communication to a doctor or a patient). When one item is indicated as being “remote” from another, this means that the two items are at least in different buildings, and may be at least one mile, ten miles, or at least one hundred miles apart. “Communicating” information means transmitting the data representing that information as electrical signals over a suitable communication channel (for example, a private or public network).

“Forwarding” an item refers to any means of getting that item from one location to the next, whether by physically transporting that item or otherwise (where that is possible) and includes, at least in the case of data, physically transporting a medium carrying the data or communicating the data.

### EXAMPLES

The invention is demonstrated further by the following illustrative examples. Parts and percentages recited herein are by weight unless otherwise specified. Temperatures are in degrees centigrade (° C.). Unless indicated otherwise, all chemicals were obtained from Sigma Chemical Company (Sigma), St. Louis, Mo., and used as received. Sample collection may be carried out using devices as described above.

Abbreviations

The following abbreviations have the meanings set forth below:

| | |
|---|---|
| CV | coefficient of variation (sd/mean) |
| g | grams, |
| μg | microgram |
| μl | microliter |
| mg | milligram |
| dl | deciliter |
| ml | milliliter |
| ng | nanogram |
| nl | nanoliter |
| PBS | phosphate buffered saline |
| BSA | bovine serum albumin |
| ANSA | 8-anilino-1-naphthalene sulfonic acid |
| RT | room temperature |
| TBS | tris buffered saline |
| HRP | horse radish peroxidase |
| min. | minute |
| sd | standard deviation |
| OPD | ortho-phenylene diamine |
| S & S | Schleicher & Schuell Inc., Keene MH |
| NFDM | non-fat dry milk |
| TMB | tetra methylbenzidine |
| dH₂O | distilled water |
| CCD | charge coupled device |

### Example 1

Assay for Cortisol

Blocking: Where indicated, blocking of membranes and ELISA plates is accomplished by treating with 3% BSA-PBS sufficient to cover the item being blocked and incubating at RT for 1 hour with shaking. The blocked item is then washed 3 times with PBS-0.05% Tween20.

Sample recollection: 4 μl of human serum samples of known concentration of cortisol are spotted onto a ¼″ disk of blocked UltraBind membrane (from Pall Corporation, Port Washington, N.Y. P/N UL083R) and dried for at least 2 hours.

Sample extraction: Extract each paper sample in 200 μl of PBS-0.5% BSA-0.08% ANSA, every other well in a pre-blocked flat bottom microplate (Nunc from VWR Scientific Products, San Francisco Calif., P/N 436110 and 442404). Cover the wells and incubate for 2 hours at RT on a shaker (speed 8) (Labline from VWR Scientific Products, Model 4625).

Addition of standards, controls and samples: Standards (0, 0.8, 1.6, 3.1, 6.25,12.5, 25 and 50 μg/dl) are made of cortisol spiked into steroid free human serum. They are parsed into aliquots and frozen at −20° C. Controls are purchased from Sigma. Controls are parsed into aliquots and frozen (Ligand Control Set, P/N L3527).

Standards, and controls are diluted 1:50 in PBS-0.5% BSA-0.08% ANSA. Add 100 μl per well of standards, controls and sample extracts to a microplate (Nunc P/N 468667 and 469949) coated with anti-cortisol antibody (East Coast Biologics, North Berwick Me., P01-92-94M-P, L/N K17 at 6 μg/ml in TBS pH 8.00). Add 50 μl per well of cortisol-HRP conjugate (from OEM Concepts Inc., Toms River N.J., P/N H6-SO1-2) diluted 1:30,000 in PBS-0.5% BSA. Incubate for 2 hours at RT on a shaker. Wash 3 times with 300 μl per well of PBS 10 mM-0.1% Tween® (Sigma, P/N P1379).

Addition of substrate: Add 100 μl per well of OPD 1 mg/ml in substrate buffer (0.05 M citric acid−0.05 M sodium phosphate pH 5.00, 1 μl per ml of H₂O₂ 30%); incubate for 15 min. in the dark. Stop the reaction by addition of 100 μl per well of HCl 1 N and read at 492 nm.

Analysis: Calibration curve: use a 4-parameter fit to plot the mean absorbance value versus the cortisol concentrations in the standards. Read the cortisol concentration of each of the samples from the calibration curve.

Results

Intra-assay precision: Three serum samples were tested 10 times each in the same run. The results are shown below in Table 1.

TABLE 1

| | 84546 | 84559 | 84562 |
|---|---|---|---|
| Liquid samples | | | |
| n = | 10 | 10 | 10 |
| mean (μg/dl) | 5.17 | 16.32 | 26.44 |
| sd (μg/dl) | 0.58 | 0.73 | 0.60 |
| CV | 11.29% | 4.48% | 2.28% |
| Samples extracted from paper (“Paper samples”) | | | |
| n = | 10 | 10 | 10 |
| mean (μg/dl) | 2.62 | 10.44 | 18.50 |
| sd (μg/dl) | 0.30 | 0.40 | 0.96 |
| CV | 11.54% | 3.87% | 5.17% |

Accuracy: Three serum samples were spiked with equal quantities of know cortisol concentration. The recovery is calculated as the percent of the test value divided by the expected concentration. The results are shown in Table 2.

TABLE 2

| | | | | | |
|---|---|---|---|---|---|
| | | Spiked | Expected | | |
| | Initial value | conc. | conc. | Test value | |
| Sample ID | (μg/dl) | (μg/dl) | (μg/dl) | (μg/dl) | Recovery |

Liquid samples

| | | | | | |
|---|---|---|---|---|---|
| 84546 | 4.8 | 4.0 | 4.4 | 5.2 | 117% |
| | 4.8 | 14.9 | 9.9 | 9.8 | 99% |
| | 4.8 | 31.0 | 17.9 | 18.4 | 103% |
| 84559 | 13.1 | 4.0 | 8.5 | 9.7 | 114% |
| | 13.1 | 14.9 | 14.0 | 15.4 | 110% |
| | 13.1 | 31.0 | 22.0 | 22.8 | 103% |
| 84562 | 25.6 | 4.0 | 14.8 | 15.3 | 103% |
| | 25.6 | 14.9 | 20.3 | 20.6 | 101% |
| | 25.6 | 31.0 | 28.3 | 25.7 | 91% |

Samples extracted from paper (“Paper samples”)

| | | | | | |
|---|---|---|---|---|---|
| 84546 | 2.9 | 4.0 | 3.5 | 2.6 | 76% |
| | 2.9 | 14.9 | 8.9 | 6.2 | 69% |
| | 2.9 | 31.0 | 17.0 | 11.7 | 69% |
| 84559 | 9.5 | 4.0 | 6.7 | 5.7 | 84% |
| | 9.5 | 14.9 | 12.2 | 9.3 | 76% |
| | 9.5 | 31.0 | 20.2 | 15.6 | 77% |
| 84562 | 16.1 | 4.0 | 10.1 | 9.5 | 95% |
| | 16.1 | 14.9 | 15.5 | 13.7 | 88% |
| | 16.1 | 31.0 | 23.6 | 19.5 | 83% |

Linearity: One serum sample was serially diluted to 7 levels with the 0 μg/dl cortisol standard. The results are shown in Table 3.

TABLE 3

| | Expected value | Test value | |
|---|---|---|---|
| | (μg/dl) | (μg/dl) | Recovery |
| Liquid samples | | | |
| 84562 | — | 24.7 | — |
| 1:2 | 12.4 | 12.1 | 98% |
| 1:3 | 8.2 | 8.3 | 100% |
| 1:4 | 6.2 | 5.8 | 94% |
| 1:6 | 4.1 | 4.5 | 109% |
| 1:8 | 3.1 | 3.5 | 112% |
| 1:12 | 2.1 | 2.2 | 107% |
| 1:16 | 1.5 | 1.5 | 98% |
| Samples extracted from paper (“Paper samples”) | | | |
| 84562 | — | 15.4 | — |
| 1:2 | 7.7 | 7.9 | 102% |
| 1:3 | 5.1 | 5.3 | 104% |
| 1:4 | 3.8 | 4.3 | 111% |
| 1:6 | 2.6 | 2.7 | 106% |
| 1:8 | 1.9 | 1.9 | 98% |
| 1:12 | 1.3 | 0.9 | 67% |
| 1:16 | 1.0 | 0.4 | 37% |

Six other serum samples were serially diluted to 3 levels with the 0 μg/dl cortisol standard. The results are shown in Table 4.

TABLE 4

| | Expected value | Test value | |
|---|---|---|---|
| | (μg/dl) | (μg/dl) | Recovery |
| Liquid samples | | | |
| 87122 | n/a | n/a | n/a |
| 1:2 | 22.7 | 22.7 | 100% |
| 1:4 | 11.4 | 10.4 | 92% |
| 1:8 | 5.7 | 4.8 | 84% |
| 87123 | n/a | n/a | n/a |
| 1:2 | 46.3 | 46.3 | 100% |
| 1:4 | 23.1 | 20.5 | 89% |
| 1:8 | 11.6 | 10.2 | 88% |
| 87126 | n/a | n/a | n/a |
| 1:2 | 46.0 | 46.0 | 100% |
| 1:4 | 23.0 | 22.3 | 97% |
| 1:8 | 11.5 | 10.6 | 92% |
| 87127 | n/a | n/a | n/a |
| 1:2 | 20.3 | 20.3 | 100% |
| 1:4 | 10.1 | 9.4 | 93% |
| 1:8 | 5.1 | 5.1 | 101% |
| 87133 | n/a | n/a | n/a |
| 1:2 | 24.6 | 24.6 | 100% |
| 1:4 | 12.3 | 10.8 | 87% |
| 1:8 | 6.2 | 5.5 | 89% |
| 87139 | n/a | n/a | n/a |
| 1:2 | 26.0 | 26.0 | 100% |
| 1:4 | 13.0 | 12.3 | 94% |
| 1:8 | 6.5 | 6.4 | 99% |
| Samples extracted from paper (“Paper samples”) | | | |
| 87122 | n/a | n/a | n/a |
| 1:2 | 13.9 | 13.9 | 100% |
| 1:4 | 6.9 | 6.8 | 99% |
| 1:8 | 3.5 | 2.8 | 82% |
| 87123 | n/a | n/a | n/a |
| 1:2 | 31.9 | 31.9 | 100% |
| 1:4 | 16.0 | 15.3 | 96% |
| 1:8 | 8.0 | 7.7 | 96% |
| 87126 | n/a | n/a | n/a |
| 1:2 | 35.0 | 35.0 | 100% |
| 1:4 | 17.5 | 18.0 | 103% |
| 1:8 | 8.7 | 8.3 | 95% |
| 87127 | n/a | n/a | n/a |
| 1:2 | 12.9 | 12.9 | 100% |
| 1:4 | 6.4 | 6.2 | 96% |
| 1:8 | 3.2 | 3.2 | 98% |
| 87133 | n/a | n/a | n/a |
| 1:2 | 16.6 | 16.6 | 100% |
| 1:4 | 8.3 | 7.5 | 90% |
| 1:8 | 4.2 | 3.7 | 88% |
| 87139 | n/a | n/a | n/a |
| 1:2 | 22.9 | 22.9 | 100% |
| 1:4 | 11.5 | 10.3 | 90% |
| 1:8 | 5.7 | 4.1 | 71% |

Reproducibility: Twelve serum samples across the range of the assay were tested 3 times over 3 days (once per day). The results are summarized in Table 5.

TABLE 5

| | Average | Range |
|---|---|---|
| Liquid samples CV | 7.3% | 2.2%-12.5% |
| Liquid samples recovery | 111% | 95%-128% |
| Paper samples CV | 9.8% | 3.8%-29.3% |
| Paper samples recovery | 70% | 62%-82% |

Cortisol stability on paper: Ten serum samples were spotted onto ¼″ disk of blocked UltraBind membrane, 4 μl sample per disk. The disks were let to dry night at RT. Two disks per sample were extracted and tested the next day-1) The remaining disks were stored in heat sealed pouches constructed from material from Steripax, Inc., Huntington Beach Calif., PET/WPPXA/FOIL/LLDPE, Product Specification 4149) containing 4A molecular sieve desiccant (Multisorb Technologies, West Seneca N.Y., P/N 02-00041AG03), and 2 other disks per sample tested on day-8. The results are summarized in Table 6.

TABLE 6

| Sample ID | Day-1 | Day-8 | % Recovery |
|---|---|---|---|
| 88078 | 9.1 | 9.1 | 100% |
| 87128 | 14.5 | 15.4 | 106% |
| 87129 | 19.3 | 18.8 | 97% |
| 87130 | 22.8 | 21.4 | 94% |
| 87132 | 18.1 | 19.3 | 107% |
| 84755 | 0.5 | 0.6 | 129% |
| 84757 | 4.2 | 4.1 | 97% |
| 84758 | 5.3 | 4.4 | 84% |
| 84760 | 7.7 | 7.7 | 99% |
| 84761 | 11.6 | 10.6 | 91% |

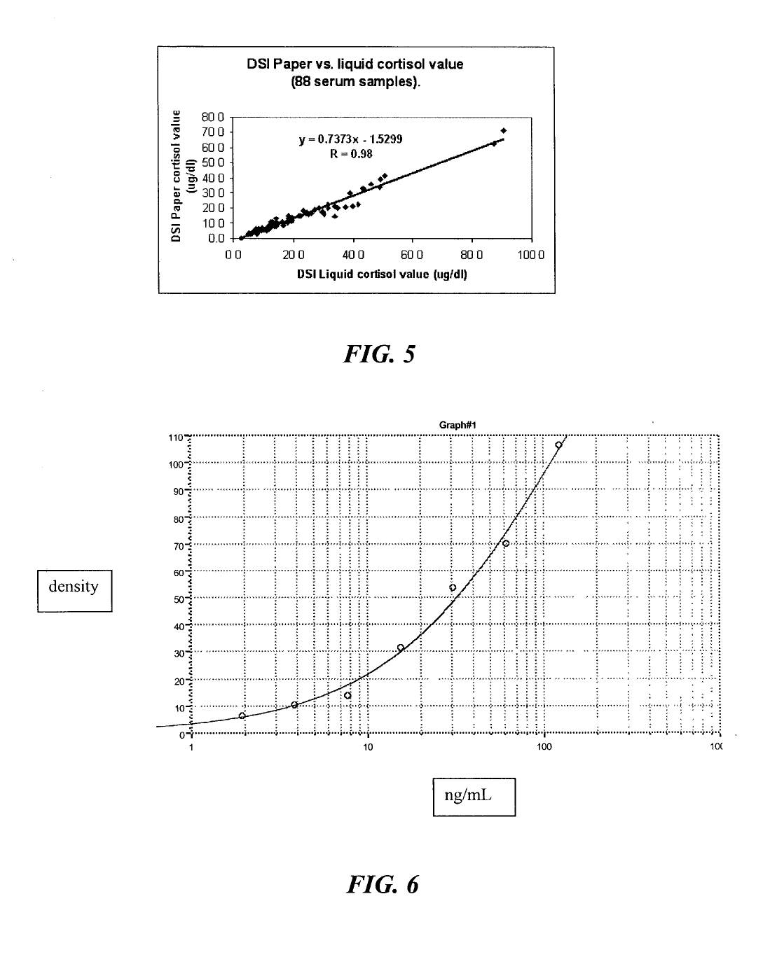

Correlation: 88 serum samples were tested as liquid and after extraction from paper. The “liquid” values were compared to the Immulite values provided by the Laboratory (New York Biologics, Inc., New York N.Y.). Immulite is manufactured by DPC, Los Angeles, Calif. The “paper” and “liquid” cortisol values from herein were compared with each other to assess the extraction efficiency. The results are shown in FIG. 5.

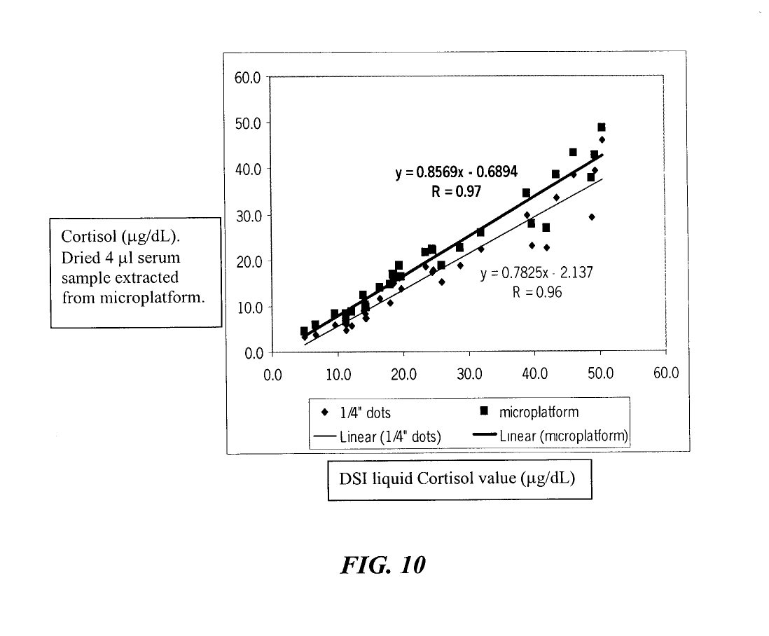

Test the analytical membrane 158 as part of the inner-housing 154 of the assay module 150 (FIG. 2A): The microplatform or analytical membrane (Ultrabind) was blocked as described above, and glued (Welldit, Devcon, Danvers, Mass. 01923) to the inner housing 154. 30 unique serum samples were spotted (4 μl per spot) to the analytical membrane attached to the inner housing of the assay module as depicted in FIG. 2A. The membranes were dried for 2 hours at RT. Cortisol was extracted from the membranes in a pre-blocked blocked 96 wells ELISA plate, shaking speed 5. The samples were spotted in parallel on free ¼″ disks of the same membrane. Extracted material was tested according to the Cortisol assay protocol. The results are shown in FIG. 10.

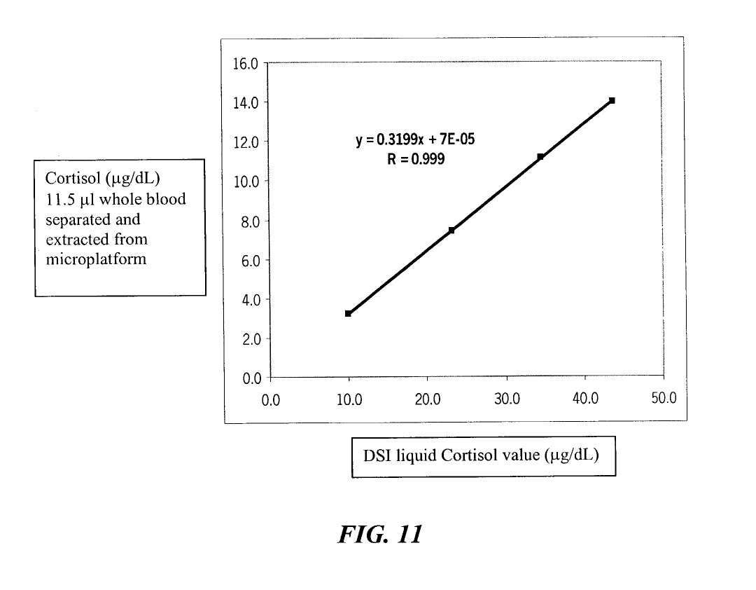

Test whole blood samples using the assay module 150 depicted in FIG. 2B: The blood separation filter (Primecare membrane, P/N PSG0002, L/N S1311G/01A Spectral Diagnostics Inc., Whitestone, Va.) used in this experiment was treated with PBS-0.1% Tween 20 for 1 hour, dried and then glued (Welldit, Devcon, Danvers, Mass. 01923) to the outer housing 152 of the device. The microplatform or analytical membrane was blocked for 1 hour in 3% BSA-PBS as described above, and glued to the inner housing 154. One whole blood sample was separated into four aliquots and spiked with varying levels of cortisol. Sufficient whole blood to fill the blood separation filter 156 (11.5 μl) was added to the blood separation filter 156 attached to the outer-housing 152 of the device. The filter 156 was then urged into contact with the microplatform 158 using a probe tip or pusher with a force of 12 g for 1 minute. Plasma separated from the whole blood was collected onto the microplatform 158. The outer housing 152 was then removed from the inner-housing 154 of the assay module 150. The microplatform 158 was allowed to dry at RT for 2 hours. In parallel, plasma was collected from a larger volume of blood using centrifugation and spotted onto ¼″ disks of the analytical membrane, 4 μl plasma per spot and dried for 2 hours at RT. Cortisol was then extracted from the membranes in 3% BSA-PBS blocked 96 wells ELISA plate as described in the cortisol assay protocol, but with the shaker set at 5. Extracted material was tested according to the Cortisol assay protocol. The results are shown in Table 7 and FIG. 11.

TABLE 7

| | | Mean Cortisol | |
|---|---|---|---|
| | | value extracted | |
| | Mean liquid | from | CV (Cortisol |
| | Cortisol value | microplatform | extracted from |
| Sample ID | (μg/dL) n = 2 | (μg/dL) n = 2 | microplatform) |
| A | 10.0 | 3.2 | 15.5% |
| B | 23.3 | 7.4 | 2.3% |
| C | 34.6 | 11.2 | 1.8% |
| D | 43.8 | 14.0 | 4.3% |

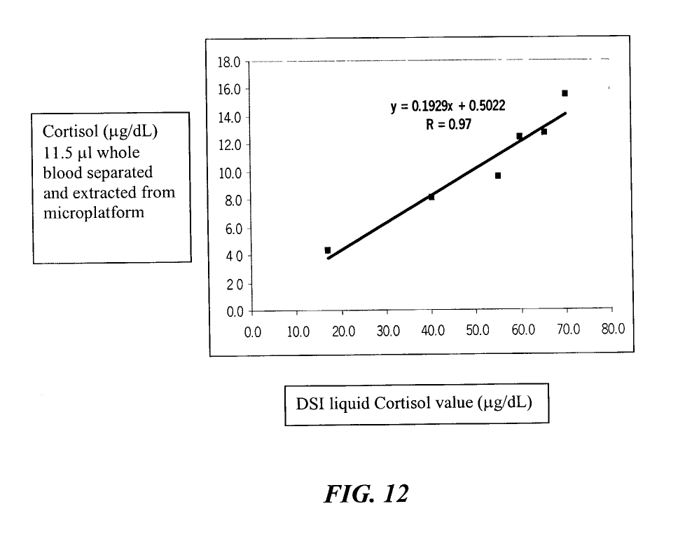

The same experiment was repeated on one whole blood sample separated into 6 aliquots, each tested 5 times. All experimental details are as described, with two exceptions: the filter 156 was untreated, and the weight used to press the pusher against the filter 156 toward the analytical membrane 158 was 59 g. The results are shown in Table 8 and FIG. 12.

TABLE 8

| | | Mean Cortisol | |
|---|---|---|---|
| | | value extracted | |
| | Mean liquid | from | CV (Cortisol |
| | Cortisol value | microplatform | extracted from |
| Sample ID | (μg/dL) n = 2 | (μg/dL) n = 5 | microplatform) |
| E | 17.0 | 4.3 | 13.0% |
| F | 40.4 | 8.1 | 13.3% |
| G | 55.2 | 9.6 | 21.9% |
| H | 60.1 | 12.4 | 18.4% |
| I | 65.6 | 12.8 | 5.4% |
| J | 70.4 | 15.5 | 8.1% |

### Example 2

Assay for LH

A) Membrane Colorimetric Reflectance Assay

In these assays, the primary antibody is coated onto the membrane, the sample is captured onto the membrane, and then the assay is developed on the membrane with a precipitating calorimetric reagent.

Coating

Monoclonal anti-LH is used to coat 0.2 micron nitrocellulose at 150 μg/ml, 2 μl per spot in 10 mM PBS pH7. The membrane is air dried for 15 min. and blocked in TBS-5% Non Fat dry milk, 0.05% Tween 20® for 1 hour at room temperature. After blocking, the membrane is washed 3 times, 15 min. per wash with wash buffer (PBS 10 mM 0.1% Tween 20®). The membrane is air dried at least 1 hour before addition of sample.

Addition of LH Sample

LH stock solution (0.5 mg/ml) is diluted to 100 ng/ml in PBS 10 mM−0.5% BSA and then serially diluted in the same buffer. Add 4 μl per well of LH solution to each antibody spot. Air-dry at least 2 hours at RT. Wash 3 times, 15 minutes per wash with wash buffer.

Conjugate

The HRP conjugated monoclonal anti-LH antibody is diluted 1:500 in PBS 10 mM−5% NFDM−0.1% Tween®. Each 2×2 cm piece of membrane is incubated in 5 mL of diluted conjugate for 1 hour at RT with shaking, and then washed 3 times with wash buffer.

Substrate

Membrane is incubated 5 minutes with 40 mL of TMB precipitating substrate, washed 2 times with dH₂O, dried, photographed and analyzed. The results are shown in FIG. 6.

B) Membrane Collection, Chemiluminescent ELISA Detection

In these assays, the sample is captured onto uncoated membrane, and is then extracted into an ELISA well that is coated with anti-LH antibody. A standard format chemiluminescent ELISA is then used to measure the LH.

Materials

Nunc 96-well MaxiSorp microplates

Monoclonal anti-human LH antibody (RDI); RDI-LH210, L/N 041598

PBS packets (Sigma), P3813, L/N 51K8207

Tween 20 (Sigma) P1379, L/N 21K0096

BSA (Sigma) A9418, L/N 80K13425

LH antigen (BiosPacific, Emeryville Calif., P/N J12020128, L/N J1840)

Normal male human serum (Bioreclamation, Hicksville N.Y.)

Monoclonal anti-human LH/FSH/HC alpha subunit—HRP conjugated

(Research Diagnostics Inc. (RDI), Flanders N.J., P/NRDI-LHA05-HRP, L/N 092601)

OPD (Sigma)

Citric acid (Sigma)

Sodium phosphate dibasic (Sigma)

H₂O₂ (Sigma)

HCl 1N (Sigma)

Coating

Prepare the monoclonal anti-LH antibody from RDI at 5 μg/ml in PBS 10 mM and coat 1 plate (100 μl per well).

Incubate overnight at RT.

Blocking

Wash the plate 3 times with 300 μl per well of PBS 10 mM-0.05% Tween®. Block with 300 μl per well of PBS 10 mM−3% BSA−0.05% Tween 20®. Incubate for 1 hour at RT on the bench (no shaking). Wash the plate 3 times with 300 μl per well of PBS 10 mM−0.05% Tween 20.

Addition of LH Samples

LH stock solution (0.5 mg/ml) is diluted to 25 ng/ml in PBS 10 mM−0.5% BSA and then serial diluted to 12.5, 6.25, 3.125, 1.6, 0.8 ng/ml in that same buffer or serum. Samples are spotted onto S&S #903 paper (4 μl per ¼″ disk); spots are dried overnight at RT. For longer storage, paper samples are stored in heat sealed pouches constructed from PET/WPPXA/FOIL/LLDPE (Steripax Product Specification 4149) containing 4A molecular sieve desiccant (Multisorb P/N 02-00041AG03). Solutions are stored overnight at 40C. Each disk is extracted the next day in 200 μl of PBS−0.5% BSA−0.05% Tween® in the coated microplate. LH liquid samples and standards are diluted 1:50 in PBS−0.5% BSA and 200 μl per well are added to the microplate. Incubate for 2 hours at RT on the shaker. Wash 3 times with 300 μl per well of PBS 10 mM−0.05% Tween®.

Conjugate

The HRP conjugated monoclonal anti-LH antibody from RDI (RDI-LHA05-HRP) is diluted 1:1000 in PBS 10 mM−0.5% BSA−0.05% Tween 20®. Add 100 μl per well of the diluted conjugate and then incubate for 1 hour at RT on the shaker. Wash 3 times with 300 μl per well of PBS 10 mM−0.05% Tween®.

Substrate

Add 100 μl Pierce supersignal ELISA Femto chemiluminescent substrate (Pierce Chemical Company, Rockford Ill., P/N 37075) and incubate 1 minute at RT. Read light development on AnalystAD (from Molecular Devices Corporation, Sunnyvale Calif.).

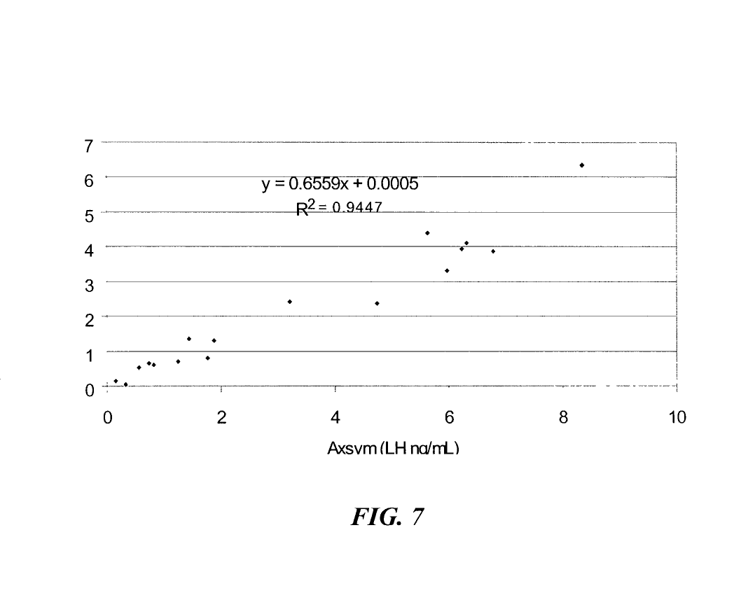

The results are shown in Table 7 and in FIG. 7. FIG. 7 depicts the results of an assay of LH serum samples conducted in accordance with the present invention and the results of a known assay (Abbott AxSym®, Abbott Laboratories, Abbott Park Ill.) performed on the same LH serum samples the Laboratory (New York Biologics, Inc., New York N.Y.).

Table 9 depicts the results of a stability study at room temperature for 7 days for a 4-microliter-serum sample on a membrane.

TABLE 9

| | | % | |
|---|---|---|---|
| [LH] ng/ml | Recovered value | recovery | CV (%) |
| 1.6 | 1.8 | 117% | 11.9% |
| 3.1 | 3.1 | 99% | 2.3% |
| 6.3 | 5.8 | 92% | 4.5% |
| 12.5 | 11.5 | 92% | 5.7% |
| | | 100% | 6.1% |

### Example 3

Assay for Glucose

Materials and Methods

Glucose oxidase (GO) (Sigma, G-9010 L/N 118H37761)

HRP (Sigma, P-6782 L/N 26H9512)

Tween 20® (Sigma, P-1379 L/N70K0117)

Cysteine-HCl (Fisher Scientific, Pittsburgh Pa., PN BP376-100, L/N996088)

BSA (Sigma, A-9418 L/N 109H0916)

D-glucose (Sigma, G-7528 L/N 69H00161)

Sodium Citrate (Sigma, S-4641 L/N99H0075)

Citric acid (Sigma, C-0706 L/N40K0893)

CUNO COAF500 from CUNO Incorporated, Meriden CT

TMB (Sigma, T-8768 L/N 87H2624)

Hydroxypropyl-beta-cyclodextrin (Sigma-Aldrich Chemical Company, St. Louis Mo., 38914-5 L/N 14303PU

Solution 1, final concentrations in 400 mM sodium citrate buffer, pH 4.0: GO 600 IU/ml; HRP 800 IU/mi; Tween 20® 0.2%; Cysteine-HCl 0.2 mg/ml; BSA 2.0 mg/ml.

Solution 2: Prepare a 300 mM solution of hydroxypropyl-beta-cyclodextrin in dH₂O. Mix well. Prepare a 10 mg/ml solution of TMB dihydrochloride in 300 mM cyclodextrin. Saturate vial with nitrogen.

Mix Solution 1 and Solution 2 in a 1:1 ratio.

Assay

Prepare a 10 mg/ml solution of D-glucose in dH₂O. Prepare 5, 2.5, 1.25, 0.625, 0.312, 0.156 mg/ml solutions by serial dilution of the above solution. Spot 2 μl of the glucose solution to be tested onto the CUNO membrane (3 replicates per concentration). Let dry at RT for at least 15 minutes. Add 2 μl of the above mixed Solutions 1 and 2. Record density with CCD.

Results

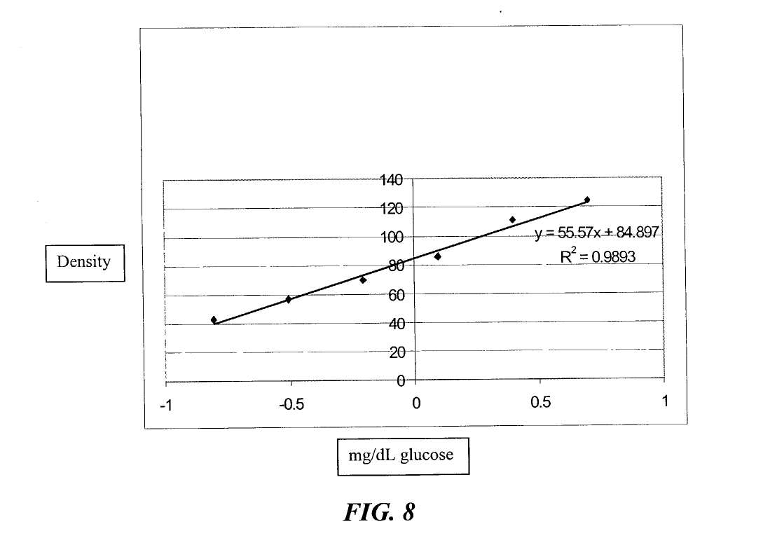

The results are shown in FIG. 8.

### Example 4

Assay for Digoxin

A) Membrane Colorimetric Reflectance Sequential-Type Assay

In these assays, the primary antibody is coated onto the membrane, the sample is captured onto the membrane and allowed to dry; sample is detected by addition of HRP-conjugated analyte, and then the assay is developed on the membrane with a precipitating calorimetric reagent.

Coating

Monoclonal anti-digoxin is used to coat 0.2-micron nitrocellulose at 300 μg/ml, 2 μl per spot in 10 mM PBS/1% sucrose pH 7. The membrane is air dried for 15 min. and blocked in TBS−5% Non Fat dry milk, 0.05% Tween 20® for 1 hour at room temperature. After blocking, the membrane is washed 3 times, 15 min. per wash with wash buffer (PBS 10 mM 0.1% Tween 20®. The membrane is air-dried at least 40 minutes before addition of sample.

Addition of LH Sample

Digoxin stock solution (0.1 mg/ml in 100% Ethanol) is diluted to target concentrations in 10 mM PBS, 0.05% Tween®, 0.5% ethanol. Add 4 μl of digoxin solution to each antibody spot. Air dry at least 2 hours at RT. Wash 3 times, 15 minutes per wash with wash buffer.

Conjugate

The HRP conjugated digoxin is diluted 0.125 μg/mL in PBS 10 mM−1% BSA−0.05% Tween®. Each 11×4 cm piece of membrane is incubated in 40 mL of diluted conjugate for 30 minutes at RT with shaking, and then washed 3 times with wash buffer.

Substrate

Membrane is incubated 10 minutes with 40 mL of TMB precipitating substrate, washed 2× with dH₂O, dried, photographed and analyzed.

Materials

Monoclonal anti-Digoxin antibody (Fitzgerald Industries International, Concord Mass., (M91281, 10-D05 Batch #133)

PBS packets (Sigma), P3813, L/N 51K8207

PBS-Tween packets (Sigma), P3563, L/N 51K8203

TBS-Tween packets (Sigma), T-6664, Lot 110K8200

BSA (Sigma) A9418, L/N 20K0944

Digoxin antigen (Sigma) D-6003, L/N 110K1536

Digoxin-HRP conjugate (BiosPacific) V56020085 L/N V1646

TMB (Sigma) T-0565, Lot 31K1389

Nitrocellulose: (S&S). BA83 0.2 micron, L/N 10402497

NFDM: (Safeway, Pleasanton Calif.)

Results

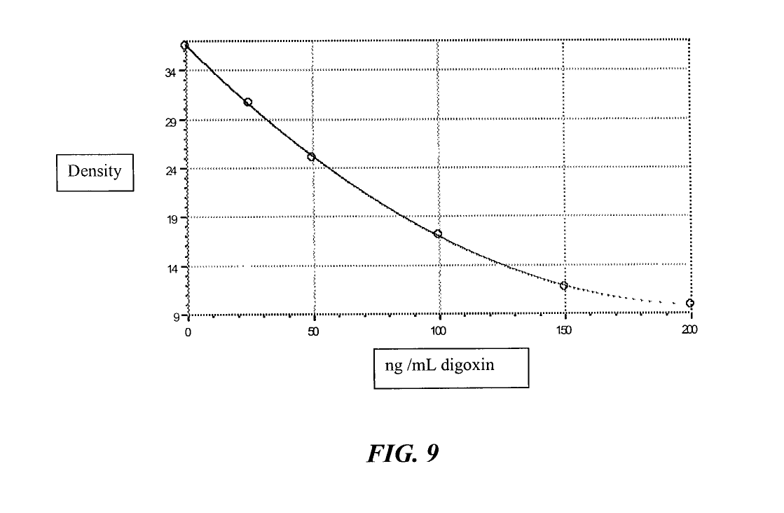

The results are shown in FIG. 9.

The aforementioned technology and devices have been shown to be capable of detecting picogram (10⁻¹² gram) quantities in 4 microliter sample volumes.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims. Furthermore, the foregoing description, for purposes of explanation, used specific nomenclature to provide a thorough understanding of the invention. However, it will be apparent to one skilled in the art that the specific details are not required in order to practice the invention. Thus, the foregoing descriptions of specific embodiments of the present invention are presented for purposes of illustration and description; they are not intended to be exhaustive or to limit the invention to the precise forms disclosed. Many modifications and variations are possible in view of the above teachings. The embodiments were chosen and described in order to explain the principles of the invention and its practical applications and to thereby enable others skilled in the art to utilize the invention.

What is claimed is:

1. A method for detecting one or more analytes, said method comprising:
a) collecting a predetermined microvolume of a fluid sample comprising said one or more analytes on a microplatform housed with a microdevice;
b) drying said microplatform; and
c) detecting the presence or amount of one or more analytes on or from said microplatform, the presence or amount thereof indicating the presence or amount of said one or more analytes.

2. A method according to claim 1 wherein said microplatform comprises a capture-moiety for each of said one or more analytes.

3. A method according to claim 1 further comprising, prior to detection step (c), extracting said one or more analytes from said microplatform.

4. The method of claim 1 further comprising, prior to detection step (c), organizing multiple microdevices having fluid samples collected therein, onto a solid-surface.

5. The method of claim 1, wherein said fluid sample is a body-fluid sample obtained from a subject, wherein said body-fluid sample is selected from the group consisting of whole-blood, plasma, serum, interstitial fluid, sweat, saliva, urine, semen, blister fluid, inflammatory exudate, body-gas and body-vapor.

6. The method of claim 5 wherein said body-fluid sample is whole-blood and said whole-blood sample is transported through a cell separating mechanism so that blood serum or blood plasma is delivered to a particular locus on said microplatform housed within the device.

7. The method of claim 5, wherein said body-fluid sample is generated by a means selected from the group consisting of a lancet, a microneedle, and skin ablation.

8. The method of claim 1, wherein said microplatform is selected from the group consisting of membranes, filters, plastic supports, silicon supports and glass supports.

9. The method of claim 1, wherein said microplatform is planar ranging in size from about 0.001 mm² to about 3 cm² or is volumetric ranging in volume from about 1 nl to about 250 microliters.

10. The method of claim 2, wherein said capture-moiety is a member of a specific binding pair.

11. The method of claim 1, wherein said detection step (c) is carried out by receptor-ligand assay.

12. The method of claim 4, wherein said solid-surface is placed into an automated detection system.

13. The method of claim 4, wherein said solid-surface contains a multiple of microdevices organized thereon selected from the group consisting of at least 6; at least 12; at least 24; at least 48; at least 96; at least 256; at least 384; at least 864; at least 1536.

14. The method of claim 1, wherein said analytes are detected by adding a detection-reagent to said microplatform.

15. The method of claim 1 wherein said microplatform has a shape selected from the group consisting of planar and three-dimensional.

16. The method of claim 4, wherein said detection-reagent is added to said platform at a different geographic location than the location of sample collection.

17. The method of claim 1, wherein the detection step is conducted at a time after fluid sample collection from about 2 hours to about 55 weeks.

18. The method of claim 1, wherein said predetermined microvolume is about 1 nanoliter to about 250 microliters.

19. The method of claim 1, wherein said sample is a non-body fluid.

20. The method of claim 1, wherein sample occupies a locus on said membrane that is about 0.001 mm² to about 3 cm².

21. A method for detecting one or more analytes in a plurality of fluid samples, comprising:
a) collecting for each fluid sample at least one predetermined microvolume of a fluid comprising said one or more analytes at a microplatform housed in a microdevice;
b) placing each of said microplatforms in a solid surface device comprising a plurality of receiving elements, each adapted to receive one of said microplatforms; and
c) detecting the presence or amount of said one or more analytes from each of said microplatforms, the presence or amount thereof being related to the presence or amount of said one or more analytes in each of said fluid samples.

22. A method according to claim 21 wherein said microplatform comprises a capture-moiety for each of said one or more analytes and said one or more analytes are detected on said microplatform.

23. A method according to claim 21 wherein said one or more analytes are non-specifically captured on said microplatform and said method comprises extracting said one or more analytes from said microplatform and examining said extracts for the presence or amount of said one or more analytes.

24. A method according to claim 21 wherein said sample is a non-body fluid sample.

25. The method of claim 21, wherein said fluid sample is a body-fluid sample obtained from a subject, wherein said body-fluid sample is selected from the group consisting of whole-blood, plasma, serum, interstitial fluid, sweat, saliva, urine, semen, blister fluid, inflammatory exudate, body-gas and body-vapor.

26. The method of claim 25, wherein said body-fluid sample is whole-blood, and said whole-blood sample is transported through a cell separating mechanism so that blood serum or blood plasma is delivered to said particular locus on said microplatform housed within the device.

27. The method of claim 25, wherein said body-fluid sample is generated by a means selected from the group consisting of a lancet, a needle, and skin ablation.

28. The method of claim 21, wherein said microplatform is selected from the group consisting of membranes, filters, plastic supports, silicon supports and glass supports.

29. The method of claim 21, wherein said microplatform is planar ranging in size from about 0.001 mm² to about 3 cm² or is volumetric ranging in volume from about 1 nl to about 250 microliters.

30. The method of claim 22, wherein said capture-moiety is a member of a specific binding pair.

31. The method of claim 21, wherein said detection step (c) is a receptor-ligand assay.

32. The method of claim 21, wherein said solid-surface device is placed into an automated detection system.

33. The method of claim 21, wherein said solid-surface device contains a multiple of microdevices organized thereon selected from the group consisting of at least 6; at least 12; at least 24; at least 48; at least 96; at least 256; at least 384; at least 864; at least 1536.

34. The method of claim 21, wherein said analytes are detected by adding a detection-reagent to said microplatform.

35. The method of claim 21, wherein said microplatform has a shape selected from the group consisting of planar and three-dimensional.

36. The method of claim 21, wherein said detection-reagent is added to said platform at a different geographic location than the location of sample collection.

37. The method of claim 21, wherein the detection step is conducted at a time after fluid sample collection from about 2 hours to about 55 weeks.

38. The method of claim 21, wherein said predetermined microvolume is about 1 nanoliter to about 250 microliters.

39. The method of claim 21, wherein said microplatform is a membrane.

40. The method of claim 21, wherein sample occupies a locus on said membrane that is about 0.001 mm² to about 3 cm².

41. The method of claim 21 further comprising drying said sample on said microplatform prior to step b).

42. A microdevice comprising:
a) a microplatform housing; and
b) a microplatform disposed within said microplatform housing, said housing being adapted such that at least a portion thereof mates with a receiving element in a solid surface device comprising a plurality of receiving elements.

43. A microdevice according to claim 42 wherein said solid surface device is a microtiter plate comprising a plurality of wells.

44. A microdevice according to claim 42 comprising a sample collection and transporting element associated with said microplatform housing wherein said microplatform is disposed within said microdevice for initiation of fluid communication with said transporting element.

45. A microdevice according to claim 44 wherein said sample collection and transporting element is contained within a second housing wherein said microplatform housing is removably mated with said second housing.

46. A microdevice according to claim 44 wherein said sample collection and transporting element comprises a capillary element and a filter.

47. A microdevice according to claim 44 wherein said sample collection and transporting element and said microplatform cooperate to take in a predetermined volume of a sample into said microdevice.

48. A microdevice according to claim 44 wherein said sample collection and transporting element comprises a sample generating means.

49. A microdevice according to claim 42 further comprising a holding element to which said microdevice is releasably attached.

50. A microdevice according to claim 49 further comprising a cap that mates with said holding element and covers said microdevice.

51. An analytical collection and transportation system comprising:
(a) a microdevice according to claim 49 and
(b) a tray comprising one or more recessed areas for housing one or more of said holding elements optionally with said microdevice releasably attached thereto.

52. An analytical collection and transportation system according to claim 51 wherein said tray further comprises one or more wells for receiving said microdevices.

53. An analytical collection and transportation system according to claim 51 further comprising a solid surface device comprising a plurality of receiving elements for receiving said microplatform housing.

54. A method for determining an analyte, said method comprising:
a) providing in combination in an assay medium a sample suspected of containing said analyte with a reagent, which comprises an antibody for said analyte having substantially all available binding sites on said antibody bound to a labeled analyte, under conditions wherein said analyte in said sample can compete off said labeled analyte from said antibody and
b) examining said antibody or said medium for the presence and or amount of said labeled analyte, the presence and/or amount thereof being related to the presence and/or amount of said analyte in said sample.

55. A method for determining an analyte in a sample suspected of containing said analyte, said method comprising:
a) collecting said analyte on a microplatform,
b) adding to said microplatform an enzyme reagent that is capable of binding to said analyte on said microplatform, and
c) examining said microplatform for the amount of enzyme activity and relating said amount to the presence and/or amount of said analyte in said sample.
